(19) United States
(12) Patent Application Publication
(10) Pub. No.: US 2026/0060287 A1
(43) Pub. Date: Mar. 5, 2026

(54) AEROSOLIZABLE COMPOSITION INCLUDING A SUBSTITUTED 3-(1-METHYLPYRROLIDIN-2-YL)PYRIDINE COMPOUND

(71) Applicant: RAI STRATEGIC HOLDINGS, INC., Winston-Salem, NC (US)

(72) Inventors: Karen V. Taluskie, Winston-Salem, NC (US); Joshua L. Watts, Winston-Salem, NC (US); Steven L. Alderman, Lewisville, NC (US); Michael A. Zawadzki, Clemmons, NC (US); Gary M. Dull, Lewisville, NC (US)

(21) Appl. No.: 19/314,205

(22) Filed: Aug. 29, 2025

Related U.S. Application Data
(60) Provisional application No. 63/689,208, filed on Aug. 30, 2024.
(60) Provisional application No. 63/863,863, filed on Aug. 14, 2025.

(51) Int. Cl. A24B 15/38; A24B 15/167; A24B 15/32; A24F 40/05; A24F 40/10; A24F 40/485; A24F 40/50; C07D 401/04; C07D 401/12

(52) U.S. Cl. CPC A24B 15/385; A24B 15/167; A24B 15/32; A24F 40/05; A24F 40/10; A24F 40/485; A24F 40/50; C07D 401/04; C07D 401/12

(57) ABSTRACT

Liquid aerosolizable compositions adapted for use in an aerosol delivery device are provided. The aerosolizable compositions include an aerosol former and a substituted 3-(1-methylpyrrolidin-2-yl)pyridine, 3-(azetidin-2-yl)pyridine, or 3-(azetidin-2-ylmethoxy)pyridine. The disclosure further provides devices incorporating such aerosolizable compositions.

### CROSS-REFERENCE TO RELATED APPLICATIONS

This application claims priority to U.S. Provisional Application No. 63/689,208, filed on Aug. 30, 2024, and claims priority to U.S. Provisional Application No. 63/863,863, filed on Aug. 14, 2025, each of which is incorporated herein by reference in its entirety and for all purposes.

### FIELD OF THE DISCLOSURE

The present disclosure relates to compositions for use in aerosol provision systems such as smoking articles designed to deliver at least one substance to a user.

### BACKGROUND

Many aerosol provision systems, and in particular, non-combustible aerosol provision systems have been proposed through the years as improvements upon, or alternatives to, smoking products that require combusting tobacco for use. These systems are generally designed to deliver at least one substance to a user, such as to satisfy a particular “consumer moment.” To this end, the substance may include constituents that impart a physiological effect on the user, a sensorial effect on the user, or both. The substance may be generally present in an aerosol-generating material that may contain one or more constituents of a range of constituents, such as active substances, flavors, aerosol-former materials and other functional materials like fillers.

Aerosol provision systems include, for example, vapor products commonly known as “electronic cigarettes,” “e-cigarettes” or electronic nicotine delivery systems (ENDS), as well as heat-not-burn products including tobacco heating products (THPs) and carbon-tipped tobacco heating products (CTHPs). Many of these products take the form of a system including a device and a consumable, and it is the consumable that includes the material from which the substance to be delivered originates. Typically, the device is reusable, and the consumable is single use (although some consumables are refillable). Therefore, in many cases, the consumable is sold separately from the device, and often in a multipack. Moreover, subsystems and some individual components of devices or consumables may be sourced from specialist manufacturers. Alternatively, aerosol provision systems may be disposable, such that once the vapor forming material is depleted, the entire device is discarded.

### BRIEF SUMMARY

The present disclosure is generally directed to liquid aerosolizable compositions for use in an aerosol delivery device. The liquid aerosolizable compositions generally comprise an aerosol former and an active agent which is a substituted 3-(1-methylpyrrolidin-2-yl)pyridine, 3-(azetidin-2-yl)pyridine, or 3-(azetidin-2-ylmethoxy)pyridine. Aerosol delivery devices comprising such aerosolizable compositions are also provided, as well as methods of producing aerosols using such aerosolizable compositions.

The present disclosure includes, without limitation, the following embodiments.

Embodiment 1: A liquid aerosolizable composition, comprising: an aerosol former; and an active agent selected from (i) a substituted 3-(1-methylpyrrolidin-2-yl)pyridine having a structure according to Formula I:

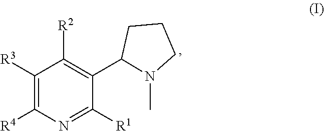

wherein:
   R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and
   wherein at least one of R¹, R², R³, and R⁴ are not hydrogen;
(ii) a substituted 3-(1-methylpyrrolidin-2-yl)pyridine having a structure according to Formula II:

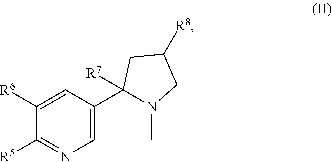

wherein R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen; and
(iii) a 3-(azetidin-2-yl)pyridine or 3-(azetidin-2-ylmethoxy)pyridine having a structure according to Formula III:

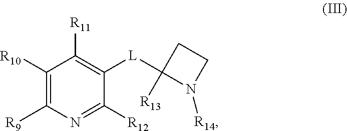

wherein L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

Embodiment 2: The composition of Embodiment 1, wherein the active agent has a structure according to Formula I, and wherein R¹, R², and R³ are each H.

Embodiment 3: The composition of Embodiment 1 or 2, wherein the active agent has a structure according to Formula I, and wherein R⁴ is optionally substituted C₁-C₆ alkyl, F, Cl, Br, OCH₃, OEt, or CN.

Embodiment 4: The composition of any one of Embodiments 1-3, wherein the active agent has a structure according to Formula I, and wherein R¹, R², and R³ are each H, and R⁴ is C₁-C₃ alkyl, such as wherein R⁴ is CH₃.

Embodiment 5: The composition of any one of Embodiments 1-4, wherein the active agent has a structure according to Formula I, and wherein the substituted 3-(1-methylpyrrolidin-2-yl)pyridine according to Formula I is racemic.

Embodiment 6: The composition of any one of Embodiments 1-4, wherein the active agent has a structure according to Formula I, and wherein the substituted 3-(1-methylpyrrolidin-2-yl)pyridine according to Formula I has an (R)-configuration or an (S)-configuration, such as wherein the substituted 3-(1-methylpyrrolidin-2-yl)pyridine has an (S)-configuration.

Embodiment 7: The composition of any one of Embodiments 1-6, wherein the composition is substantially free of 3-(1-methylpyrrolidin-2-yl)pyridine, such as wherein the composition is completely free of 3-(1-methylpyrrolidin-2-yl)pyridine.

Embodiment 8: The composition of any one of Embodiments 1-7, wherein the active agent has a structure according to Formula I, and wherein the substituted 3-(1-methylpyrrolidin-2-yl)pyridine is present in an amount from about 0.1 to about 5% by weight of the composition, calculated as the free base and based on the total weight of the composition, such as about 0.1 to about 1.0% by weight or about 0.1 to 0.75% by weight or about 0.1 to about 0.5% by weight.

Embodiment 9: The composition of any one of Embodiments 1-8, wherein the composition is substantially free of acid components; optionally, wherein the composition is completely free of acid components.

Embodiment 10: The composition of any one of Embodiments 1-8, further comprising an organic acid.

Embodiment 11: The composition of Embodiment 10, wherein the active agent has a structure according to Formula I, and wherein at least a portion of the substituted 3-(1-methylpyrrolidin-2-yl)pyridine is present in the form of a salt with the organic acid.

Embodiment 12: The composition of Embodiment 10 or 11, wherein the active agent has a structure according to Formula I, and wherein at least a portion of the substituted 3-(1-methylpyrrolidin-2-yl)pyridine is present in the form of a salt with tartaric acid.

Embodiment 13: The composition of Embodiment 10 or 11, wherein the active agent has a structure according to Formula I, and wherein at least a portion of the substituted 3-(1-methylpyrrolidin-2-yl)pyridine is present in the form of a salt with lactic acid, levulinic acid, or a combination thereof.

Embodiment 14: The composition of any one of Embodiments 10-13, wherein the active agent has a structure according to Formula I, and wherein the organic acid and the substituted 3-(1-methylpyrrolidin-2-yl)pyridine are present in a molar ratio in a range from about 1:10 to about 2:1, such as about 1:2 to about 2:1, or about 0.9:1 to about 1.1:1, such as wherein the molar ratio is 1:10 to about 1:1 or about 1:10 to about 1:1.1 or about 1:5 to about 1:1.2 or about 1:4 to about 1:1.5.

Embodiment 15: The composition of any one of Embodiments 1-14, wherein the aerosol former comprises one or more polyhydric alcohols, water, or a combination thereof; optionally, wherein the one or more polyhydric alcohols is selected from the group consisting of glycerol, propylene glycol, 1,3-propanediol, diethylene glycol, triethylene glycol, a polyethylene glycol, and combinations thereof, such as wherein the polyhydric alcohol is selected from the group consisting of glycerol, propylene glycol, and mixtures thereof, or wherein the aerosol former comprises propylene glycol and glycerol.

Embodiment 16: The composition of Embodiment 15, wherein the aerosol former comprises propylene glycol and glycerol in a ratio by weight from about 1:3 to about 3:1; such as about 1:1 to about 2.5:1 or about 1.1:1 to about 2:1 or about 1.1:1 to about 1.5:1.

Embodiment 17: An aerosol delivery device comprising a housing enclosing a chamber containing the liquid aerosolizable composition of any one of Embodiments 1 to 16.

Embodiment 18: An aerosol delivery device comprising: a housing enclosing a chamber containing the liquid aerosolizable composition of any one of Embodiments 1 to 16; an atomizer in fluid communication with the chamber and configured to aerosolize the liquid aerosolizable composition to form an aerosol; and an aerosol pathway positioned to carry the aerosol to a mouth-end of the aerosol delivery device.

Embodiment 19: The aerosol delivery device of Embodiment 18, further comprising a power source electronically connected to the atomizer.

Embodiment 20: The aerosol delivery device of Embodiment 19, further comprising a controller configured to control the power transmitted by the power source to the atomizer.

Embodiment 21: The aerosol delivery device of any one of Embodiments 18 to 20, wherein the atomizer comprises a heating element or a piezoelectric element.

Embodiment 22: A process for forming an aerosol, the process comprising aerosolizing a liquid aerosolizable composition of any one of Embodiments 1 to 16.

Embodiment 23: An aerosol comprising: an aerosol former; and an active agent selected from:
(i) a substituted 3-(1-methylpyrrolidin-2-yl)pyridine having a structure according to Formula I:

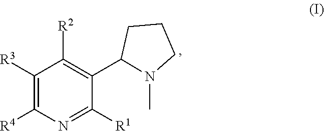

wherein:
R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted, and at least one of R¹, R², R³, and R⁴ are not hydrogen;
(ii) a substituted 3-(1-methylpyrrolidin-2-yl)pyridine having a structure according to Formula II:

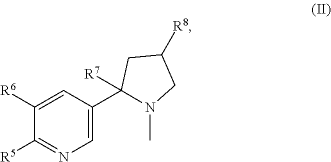

wherein R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen; and
(iii) a 3-(azetidin-2-yl)pyridine or 3-(azetidin-2-ylmethoxy)pyridine having a structure according to Formula III:

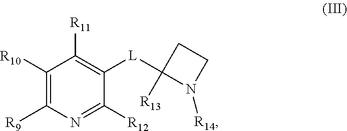

wherein L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

Embodiment 24: A liquid aerosolizable composition, comprising: an aerosol former; and
a substituted 3-(1-methylpyrrolidin-2-yl)pyridine having a structure according to Formula II:

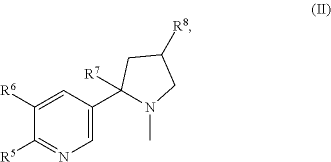

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C₁-C₃ alkyl; and
   at least one of R⁷ and R⁸ is not hydrogen.

Embodiment 25: The composition of Embodiment 24, wherein: R¹ is hydrogen or C1-C3 alkyl; and R² is H.

Embodiment 26: The composition of Embodiment 25, wherein R¹ is H, and wherein: R³ is H and R⁴ is CH₃; R³ is CH₃ and R⁴ is H; or R³ and R⁴ are both CH₃.

Embodiment 27: The composition of Embodiment 26, wherein R³ is H and R⁴ is CH₃.

Embodiment 28: The composition of Embodiment 26, wherein R³ is CH₃ and R⁴ is H.

Embodiment 29: The composition of Embodiment 25, wherein R¹ is CH₃, and wherein: R³ is H and R⁴ is CH₃; or R³ is CH₃ and R⁴ is H.

Embodiment 30: The composition of Embodiment 29, wherein R³ is CH₃ and R⁴ is H.

Embodiment 31: The composition of any one of Embodiments 24-30, wherein the substituted 3-(1-methylpyrrolidin-2-yl)pyridine is racemic.

Embodiment 32: The composition of any one of Embodiments 24-41, wherein the substituted 3-(1-methylpyrrolidin-2-yl)pyridine has an (R)-configuration or an (S)-configuration at the 2′ position, such as wherein the substituted 3-(1-methylpyrrolidin-2-yl)pyridine has an (S)-configuration at the 2′ position.

Embodiment 33: The composition of any one of Embodiments 24-32, wherein the substituted 3-(1-methylpyrrolidin-2-yl)pyridine has a calculated log P of about 1 or greater, such as from about 1.2 to about 1.7.

Embodiment 34: The composition of any one of Embodiments 24-33, wherein the composition is substantially free of 3-(1-methylpyrrolidin-2-yl)pyridine, such as wherein the composition is completely free of 3-(1-methylpyrrolidin-2-yl)pyridine.

Embodiment 35: The composition of any one of Embodiments 24-34, wherein the substituted 3-(1-methylpyrrolidin-2-yl)pyridine is present in an amount from about 0.1 to about 5% by weight of the composition, calculated as the free base and based on the total weight of the composition, such as about 0.1 to about 1.0% by weight or about 0.1 to 0.75% by weight or about 0.1 to about 0.5% by weight.

Embodiment 36: The composition of any one of Embodiments 24-35, wherein the composition is substantially free of acid components; optionally, wherein the composition is completely free of acid components.

Embodiment 37: The composition of any one of Embodiments 24-36, further comprising an organic acid.

Embodiment 38: The composition of Embodiment 37, wherein at least a portion of the substituted 3-(1-methylpyrrolidin-2-yl)pyridine is present in the form of a salt with the organic acid.

Embodiment 39: The composition of Embodiment 37 or 38, wherein at least a portion of the substituted 3-(1-methylpyrrolidin-2-yl)pyridine is present in the form of a salt with tartaric acid.

Embodiment 40: The composition of Embodiment 37 or 38, wherein at least a portion of the substituted 3-(1-methylpyrrolidin-2-yl)pyridine is present in the form of a salt with lactic acid, levulinic acid, or a combination thereof.

Embodiment 41: The composition of any one of Embodiments 37-40, wherein the organic acid and the substituted 3-(1-methylpyrrolidin-2-yl)pyridine are present in a molar ratio in a range from about 1:10 to about 2:1, such as about 1:2 to about 2:1, or about 0.9:1 to about 1.1:1, such as wherein the molar ratio is 1:10 to about 1:1 or about 1:10 to about 1:1.1 or about 1:5 to about 1:1.2 or about 1:4 to about 1:1.5.

Embodiment 42: The composition of any one of Embodiments 24-41, wherein the aerosol former comprises one or more polyhydric alcohols, water, or a combination thereof; optionally, wherein the one or more polyhydric alcohols is selected from the group consisting of glycerol, propylene glycol, 1,3-propanediol, diethylene glycol, triethylene glycol, a polyethylene glycol, and combinations thereof, such as wherein the polyhydric alcohol is selected from the group consisting of glycerol, propylene glycol, and mixtures thereof, or wherein the aerosol former comprises propylene glycol and glycerol.

Embodiment 43: The composition of Embodiment 42, wherein the aerosol former comprises propylene glycol and glycerol in a ratio by weight from about 1:3 to about 3:1; such as about 1:1 to about 2.5:1 or about 1.1:1 to about 2:1 or about 1.1:1 to about 1.5:1.

Embodiment 44: An aerosol delivery device comprising: a housing enclosing a chamber containing the liquid aerosolizable composition of any one of Embodiments 24 to 43.

Embodiment 45: An aerosol delivery device comprising: a housing enclosing a chamber containing the liquid aerosolizable composition of any one of Embodiments 24 to 43; an atomizer in fluid communication with the chamber and configured to aerosolize the liquid aerosolizable composition to form an aerosol; and an aerosol pathway positioned to carry the aerosol to a mouth-end of the aerosol delivery device.

Embodiment 46: The aerosol delivery device of Embodiment 45, further comprising a power source electronically connected to the atomizer.

Embodiment 47: The aerosol delivery device of Embodiment 46, further comprising a controller configured to control the power transmitted by the power source to the atomizer.

Embodiment 48: The aerosol delivery device of any one of Embodiments 45 to 47, wherein the atomizer comprises a heating element or a piezoelectric element.

Embodiment 49: A process for forming an aerosol, the process comprising aerosolizing a liquid aerosolizable composition of any one of Embodiments 24 to 43.

Embodiment 50: An aerosol comprising: an aerosol former; and a substituted 3-(1-methylpyrrolidin-2-yl)pyridine having a structure according to Formula II:

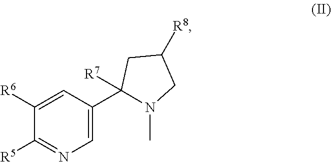

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C₁-C₃ alkyl; and
   at least one of R⁷ and R⁸ is not hydrogen.

Embodiment 51: The composition of Embodiment 1, wherein the active agent has a structure according to Formula IL, optionally wherein R⁵ is optionally substituted C₁-C₆ alkyl, F, Cl, Br, OCH₃, OCH₂CH₃, or CN; and R⁶ is H, or optionally wherein R⁵ is H; and R⁶ is optionally substituted C₁-C₆ alkyl, F, Cl, Br, OCH₃, OCH₂CH₃, or CN.

Embodiment 52: The composition of Embodiment 1, wherein the active agent has a structure according to Formula III, optionally wherein R⁹ is H or CH₃; and R¹⁰, R¹¹, and R¹² are each H.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

### BRIEF DESCRIPTION OF THE DRAWINGS

Having thus described aspects of the disclosure in the foregoing general terms, reference will now be made to the accompanying figures, which are not necessarily drawn to scale, and wherein:

FIG. 1 is a block diagram of an aerosol provision system according to non-limiting embodiments of the present disclosure.

FIGS. 2 and 3 illustrate an aerosol provision system in the form of a vapor product, according to a non-limiting embodiment of the present disclosure.

FIG. 4 illustrates a nebulizer that may be used to implement an aerosol generator of an aerosol provision system, according to a non-limiting embodiment of the present disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise.

Unless otherwise defined, reference to the aerosolizable composition being “substantially free” of a component herein means that the component has not been intentionally added, and is therefore not present beyond trace amounts that may be present e.g., as an impurity in another component. For example, some embodiments can be characterized as having less than 0.01% by weight of the component, or less than 0.001% by weight of the component, or less than 0.0001%, or even 0% by weight of the component.

The present disclosure is generally directed to aerosolizable compositions for use in an aerosol delivery device, and to various aerosol delivery devices comprising such aerosolizable compositions. The aerosolizable compositions are capable of generating aerosol, for example when heated, irradiated, or energized in any suitable manner. Accordingly, the disclosure provides aerosolizable compositions (also referred to herein as “aerosol generating materials” or “aerosol precursor compositions”) adapted for use in an aerosol delivery device. Such aerosolizable compositions and aerosol delivery devices comprising them are described further herein below.

### Aerosolizable Compositions

Aerosolizable compositions as disclosed herein comprise an aerosol former and a substituted 3-(1-methylpyrrolidin-2-yl)pyridine. Each of these components is described further herein below.

### Aerosol Former

Aerosol precursor compositions as disclosed herein comprise an aerosol former. Generally, the aerosol former includes one or more constituents capable of forming an aerosol, for example, by thermal or mechanical vaporization. Non-limiting examples of suitable classes of aerosol formers include water, polyhydric alcohols, polysorbates, sorbitan esters, fatty acids, fatty acid esters, triacetin, waxes, sugar alcohols, and combinations thereof. The particular choice of aerosol former(s) may depend on factors such as the method of aerosol formation, the appearance and volume of the aerosol, the desired density of the aerosol, and the like.

The total amount of aerosol former(s) in the composition can vary, but is typically up to about 99.9% by weight, and some embodiments are characterized by an aerosol former content of about 50% to about 99% by weight, about 60% to about 95% by weight, or about 70% to about 90% weight, based on the total weight of the composition.

In some embodiments, the aerosol former may include one or more polyhydric alcohols. Examples of polyhydric alcohols include glycerol (also referred to as glycerin herein), propylene glycol, and other glycols such as 1,3-propanediol, diethylene glycol, triethylene glycol, and polyethylene glycols (e.g., PEG molecules with weight average molecular weight range of about 200 to about 2,000 Da). In some embodiments, the aerosol former comprises one or more polyhydric alcohols. In some embodiments, the one or more polyhydric alcohols are selected from the group consisting of glycerol, propylene glycol, 1,3-propanediol, diethylene glycol, triethylene glycol, a polyethylene glycol, and combinations thereof. In some embodiments, the polyhydric alcohol is selected from the group consisting of glycerol, propylene glycol, and mixtures thereof. In some embodiments, the aerosol forming composition is substantially free of propylene glycol. In some embodiments, the aerosol forming composition is substantially free of glycerin.

In some embodiments, the aerosol former comprises a combination of propylene glycol and glycerol. The relative amounts of propylene glycol and glycerol may vary and may be expressed as a weight ratio. In some embodiments, the aerosol former comprises propylene glycol and glycerol in a ratio by weight from about 1:3 to about 3:1, such as about 1:3, about 1:2, about 1:1, about 2:1, or about 3:1, or such as about 1:1 to about 2.5:1 or about 1.1:1 to about 2:1 or about 1.1:1 to about 1.5:1. In some embodiments, use of propylene glycol in a greater amount than glycerol can improve the sensory characteristics of aerosol produced using the aerosolizable composition.

In some embodiments, the aerosol former comprises water. In some embodiments, the aerosol former comprises water and one or more polyhydric alcohols. In some embodiments, the aerosol former comprises water and one or more polyhydric alcohols selected from the group consisting of glycerol and propylene glycol. In embodiments where the aerosol former comprises water and a polyhydric alcohol, the ratio of water to polyhydric alcohol may vary. For example, the ratio of water to polyhydric alcohol may be from about 1:10 to about 10:1 by weight, such as about 1:10, 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1. In embodiments ratio by weight of water to polyhydric alcohol is from about 1:9 to about 9:1. In embodiments ratio by weight of water to polyhydric alcohol is from about 1:1 to about 9:1.

In some embodiments, the aerosol former further comprises one or more polysorbates, one or more sorbitan esters, one or more fatty acids, one or more fatty acid esters, triacetin, one or more waxes, one or more sugar alcohols, or combinations thereof. Examples of polysorbates include, but are not limited to, Polysorbate 60 (polyoxyethylene (20) sorbitan monostearate, Tween® 60) and Polysorbate 80 (polyoxyethylene (20) sorbitan monooleate; Tween® 80). Examples of sorbitan esters include, but are not limited to, sorbitan monolaurate, sorbitan monostearate (Span® 60), sorbitan monooleate (Span® 20), and sorbitan tristearate (Span® 65). Examples of fatty acids include, but are not limited to, butyric, propionic, valeric, oleic, linoleic, stearic, myristic, and palmitic acids. Examples of fatty acid esters include, but are not limited to, alkyl esters, monoglycerides, diglycerides, and triglycerides. Examples of monoglycerides include monolaurin and glycerol monostearate. Examples of triglycerides include triolein, tripalmitin, tristearate, glycerol tributyrate, and glycerol trihexanoate. Examples of waxes include, but are not limited to, carnauba, beeswax, candelilla. Examples of sugar alcohols include, but are not limited to, sorbitol, erythritol, mannitol, maltitol, isomalt, and xylitol. In some embodiments, the aerosol former further comprises erythritol, meso-erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, propylene carbonate, or combinations thereof.

### Active Agents

### Substituted 3-(1-methylpyrrolidin-2-yl)pyridine

In some embodiments, the liquid aerosol precursor compositions of the disclosure comprise a substituted 3-(1-methylpyrrolidin-2-yl)pyridine. As used herein, the term “substituted 3-(1-methylpyrrolidin-2-yl)pyridine” refers to a compound having a 3-(1-pyrrolidin-2-yl)pyridine) scaffold and bearing one or more non-hydrogen substituents on the pyridine ring. In preferred embodiments, the substituted 3-(1-methylpyrrolidin-2-yl)pyridine present is not synthetically derived from nicotine.

In some embodiments, the compositions of the disclosure can be characterized as completely free or substantially free of nicotine (3-(1-methylpyrrolidin-2-yl)pyridine). By “substantially free” it is meant that no nicotine has been intentionally added, beyond trace amounts that may be present e.g., as an impurity in another component, including as a minor impurity in the substituted 3-(1-methylpyrrolidin-2-yl)pyridine. For example, some embodiments can be characterized as having less than 0.01% by weight of nicotine, or less than 0.001% by weight of nicotine, or less than 0.0001%, or even 0% by weight of nicotine, calculated as the free base and based on the total weight of the composition. In some embodiments the composition is completely free of (R)-, (S)-, and (R/S)-3-(1-methylpyrrolidin-2-yl)pyridine (e.g., having 0% by weight of nicotine, including racemic nicotine and nicotine enantiomers, calculated as the free base and based on the total weight of the composition).

In some embodiments, the substituted 3-(1-methylpyrrolidin-2-yl)pyridine has a structure according to Formula I:

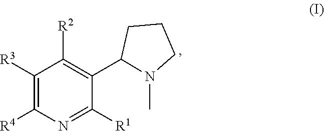

wherein:
R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen.

Substituted 3-(1-methylpyrrolidin-2-yl)pyridines with various R¹, R², R³, and R⁴ substituents have been reported previously. See for example, U.S. Pat. Nos. 4,321,387, 4,155,909; 5,015,741, 5,138,062, and 5,703,100, each of which is incorporated by reference herein and describe such substituted 3-(1-methylpyrrolidin-2-yl)pyridines, their synthesis, and pharmacological properties. Substituted 3-(1-methylpyrrolidin-2-yl)pyridines and their pharmacological profiles have also been disclosed in Wang et al., Drug Development Research 1998, Volume 45, Issue 1, Pages 10-16; and Dukat et al. European Journal of Medicinal Chemistry 1999, 34(1): 31-40.

In some embodiments, R¹, R², and R⁴ are each H, and R³ is a non-hydrogen substituent. In some embodiments, R³ is optionally substituted C₁-C₆ alkyl, F, Cl, Br, OMe, OEt, or CN. In some embodiments, R³ is Me or Et. In some embodiments, R³ is F.

In some embodiments, R¹, R², and R³ are each H, and R⁴ is a non-hydrogen substituent.

In some embodiments, R⁴ is optionally substituted C₁-C₆ alkyl, F, Cl, Br, OMe, OEt, or CN.

In some embodiments, R¹, R², and R³ are each H, and R⁴ is optionally substituted C1-C6 alkyl, F, Cl, Br, OCH₃, OEt, or CN.

In some embodiments, R¹, R², and R³ are each H, and R⁴ is C₁-C₃ alkyl.

In some embodiments, R¹, R², and R³ are each H, and R⁴ is Me. In such embodiments, the compound of Formula I may be referred to as 2-methyl-5-(1-methylpyrrolidin-2-yl)pyridine.

The pharmacology of 2-methyl-5-(1-methylpyrrolidin-2-yl)pyridine has been reported in, for example, Dukat et al. European Journal of Medicinal Chemistry, Volume 31, Issue 11, 1996, Pages 875-888 (incorporated herein by reference), and the pharmacological profile of the (S)-enantiomer of 2-methyl-5-(1-methylpyrrolidin-2-yl)pyridine in the form of the benzoate salt (CAS 2861225-70-7; referred to as Imotine™) is discussed in Carmines et al, Poster #6; 76^{th} TSRC Conference 2023, Norfolk, VA, USA).

In some embodiments, the substituted 3-(1-methylpyrrolidin-2-yl)pyridine has a structure according to Formula II:

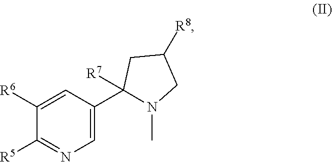

wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and at least one of R⁷ and R⁸ is not hydrogen.

Certain substituted 3-(1-methylpyrrolidin-2-yl)pyridines with various R⁵, R⁶, R⁷, and R⁸ substituents have been reported previously. See for example, U.S. Pat. Nos. 4,321,387, 4,155,909; 5,015,741, 5,138,062, and 5,703,100, each of which is incorporated by reference herein and describe example substituted 3-(1-methylpyrrolidin-2-yl)pyridines, their synthesis, and pharmacological properties. Certain substituted 3-(1-methylpyrrolidin-2-yl)pyridines and their pharmacological profiles have also been disclosed in Wang et al., Drug Development Research 1998, Volume 45, Issue 1, Pages 10-16; Dukat et al. European Journal of Medicinal Chemistry 1999, 34(1): 31-40; Lin et al., J. Med. Chem. (1994), 37, 3542-3553.

In some embodiments, R⁵ and R⁶ are H; R⁷ is CH₃; and R⁸ is H. In such embodiments, the compound of Formula II may be referred to as 3-(1,2-dimethylpyrrolidin-2-yl)pyridine, or 2′-methyl-5-(1-methylpyrrolidin-2-yl)pyridine, and has a structure:

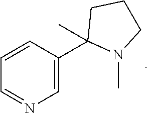

The compound 3-(1,2-dimethylpyrrolidin-2-yl)pyridine is known in the literature, and has a Chemical Abstracts Registry Number of 220650-38-4. The synthesis of this compound has been reported in Rouchaud et al., J Het Chem 2012, 49(1), 161-166; Wang et al., Drug Dev Res 1998, 45(1), 10-16; Secor et al., Tetrahedron Lett. (1981), 22(33), 3151-3154; US Patent Application Publication No. 2013/0157995; PCT Application Publication No. WO2012/031220; and U.S. Pat. No. 9,440,948, each of which are incorporated by reference herein with respect to the synthesis of 3-(1,2-dimethylpyrrolidin-2-yl)pyridine.

In some embodiments, R⁵ and R⁶ are H; R⁷ is H; and R⁸ is CH₃. In such embodiments, the compound of Formula II may be referred to as 3-(1,4-dimethylpyrrolidin-2-yl)pyridine, or 4′-methyl-5-(1-methylpyrrolidin-2-yl)pyridine, and has a structure:

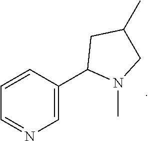

The compound 3-(1,4-dimethylpyrrolidin-2-yl)pyridine is known in the literature, has a Chemical Abstracts Registry Number of 74805-00-8, and is commercially available from, for example, Enamine Stock Building Blocks and Aurora Building Blocks. The synthesis of this compound has been reported in U.S. Pat. No. 9,440,948; and EP Patent No. 559495, each of which are incorporated by reference herein with respect to the synthesis of 3-(1,4-dimethylpyrrolidin-2-yl)pyridine.

In some embodiments, R⁵ is CH₃; R⁶ is H; R⁷ is CH₃; and R⁸ is H. In such embodiments, the compound of Formula II may be referred to as 5-(1,2-dimethylpyrrolidin-2-yl)-2-methylpyridine, and has a structure:

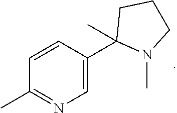

The compound 5-(1,2-dimethylpyrrolidin-2-yl)-2-methylpyridine may be readily synthesized according to known reactions. For example, commercially available 2-methyl-5-(2-methylpyrrolidin-2-yl)pyridine (Chemical Abstracts Registry Number of 1528955-30-7; Aurora Building Blocks, Adlab Chemicals Building Blocks) can be N-methylated with formaldehyde and formic acid, or alternatively with formaldehyde and a reducing agent such as sodium cyanoborohydride to afford 5-(1,2-dimethylpyrrolidin-2-yl)-2-methylpyridine. Alternatively, 5-(1,2-dimethylpyrrolidin-2-yl)-2-methylpyridine may be synthesized by lithiation of 2-methyl-5-bromopyridine, reaction of the lithiated pyridine with N-methylpyrrolidone, and addition of methyl lithium to the perchlorate salt of the resulting imine. This reaction sequence is shown below in Scheme 1.

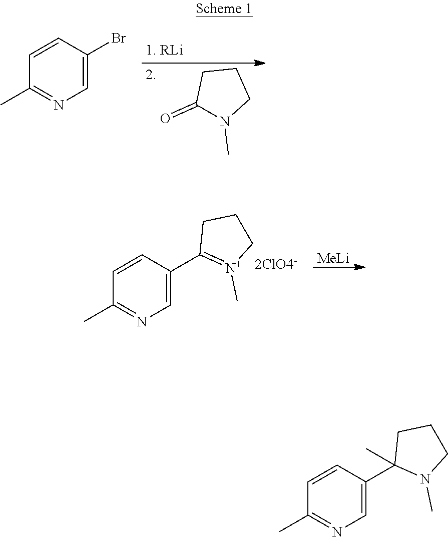

In some embodiments, R⁵ is H or CH₃, R⁶ is H, R⁷ is H or CH₃, and R⁹ is H or CH₃, provided that at least one of R⁷ and R⁸ is CH₃. Accordingly, in some embodiments, the substituted 3-(1-methylpyrrolidin-2-yl)pyridine has a structure selected from

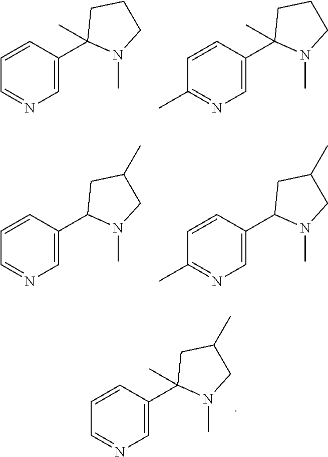

In some embodiments, R⁵ is H, R⁶ is F, CH₃, or OCH₃, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from

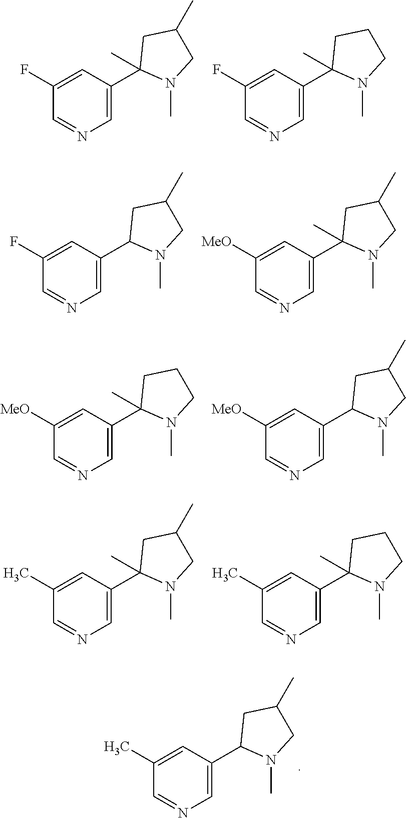

A substituted 3-(1-methylpyrrolidin-2-yl)pyridine and bearing one or more substituents on the pyrrolidine ring as described herein may be present as a single enantiomer or as a mixture of enantiomers. In some embodiments, the substituted 3-(1-methylpyrrolidin-2-yl)pyridine bearing one or more substituents on the pyrrolidine ring is present in racemic form, meaning there are equal amounts of (R)- and (S)-enantiomers present. In some embodiments, the composition comprises unequal amounts of (R)- and (S)-enantiomer (i.e., is enriched in either the (R)- or (S)-enantiomer. In some embodiments, the composition predominantly comprises the substituted 3-(1-methylpyrrolidin-2-yl)pyridine bearing one or more substituents on the pyrrolidine ring in the (R)-configuration, for example, about 90% or more of the total quantity of substituted 3-(1-methylpyrrolidin-2-yl)pyridine bearing one or more substituents on the pyrrolidine ring present is in the (R)-configuration. In some embodiments, the composition predominantly comprises the substituted 3-(1-methylpyrrolidin-2-yl)pyridine bearing one or more substituents on the pyrrolidine ring in the (S)-configuration, for example, about 90% or more of the total quantity of substituted 3-(1-methylpyrrolidin-2-yl)pyridine present is in the (S)-configuration. In some embodiments, the composition comprises 95% or more of the (S)-configuration of the substituted 3-(1-methylpyrrolidin-2-yl)pyridine, based on the total amount of substituted 3-(1-methylpyrrolidin-2-yl)pyridine present.

In some embodiments, the substituted 3-(1-methylpyrrolidin-2-yl)pyridine bearing one or more substituents on the pyrrolidine ring is non-racemic, and has one of the following structures:

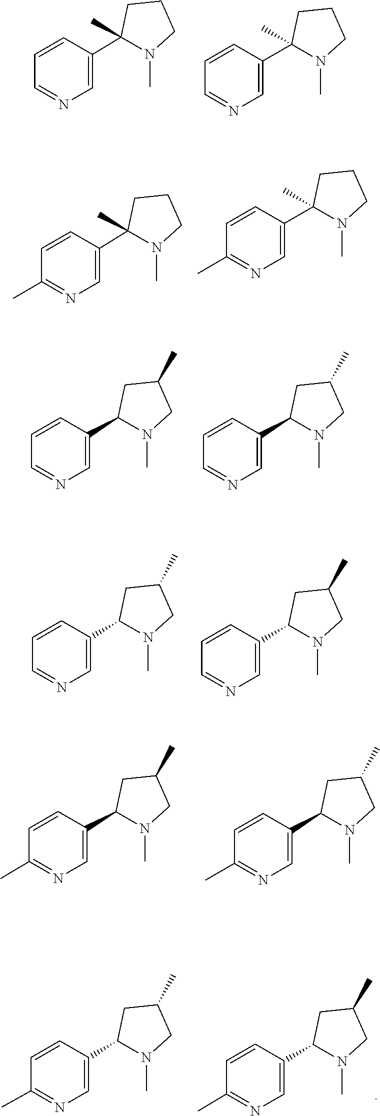

In some embodiments, the substituted 3-(1-methylpyrrolidin-2-yl)pyridine bearing one or more substituents on the pyrrolidine ring is non-racemic, and has a structure selected from:

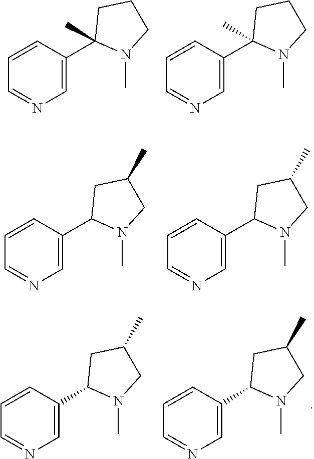

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of various substituted 3-(1-methylpyrrolidin-2-yl)pyridines such as those described herein has been reported in, for example, Lin et al., J. Med. Chem., 1994, 37, 3542-3553; Dukat et al. European Journal of Medicinal Chemistry, 31(11), 1996, 875-888; U.S. Pat. No. 9,440,948; Wang et al., Drug Dev Res 1998, 45(1), 10-16 (each of which is incorporated herein by reference), among many others. Generally, small substituents such as methyl groups are well tolerated at the 2′ or 4′ positions of the nicotine pyrrolidine ring, and small substituents such as alkyl, halogen, alkoxy, and the like are well tolerated at the 5 or 6 position of the nicotine pyridine ring. For example, 3-(1-methylpyrrolidin-2-yl)pyridines bearing a methyl substituent at the 2′ or 4′ position are equipotent or even more potent than nicotine with respect to binding affinity to the nicotinic acetylcholine receptor, and are expected to preserve the pharmacological effects of nicotine in vivo. See, for example U.S. Pat. No. 5,278,176, Lin et al., J. Med. Chem., 1994, 37, 3542-3553, and Wang et al., Drug Dev Res 1998, 45(1), 10-16.

Without wishing to be bound by any particular theory, it is believed that certain substitutions for hydrogen on the pyridine ring and/or pyrrolidine rings of the substituted 3-(1-methylpyrrolidin-2-yl)pyridine compound preserve the general pharmacological profile and physiological effects of nicotine while offering the potential for one or more of greater potency, longer half-life, reduced product consumption, more rapid and/or complete absorption, greater bioavailability, and the like. Particularly, it is believed that in some embodiments, substituted 3-(1-methylpyrrolidin-2-yl)pyridines of the disclosure are readily absorbed through membranes such as pulmonary alveoli, oral mucosa, and the like by virtue of their lipophilicity. Lipophilicity is conveniently measured in terms of log P, the partition coefficient of a molecule between a lipophilic phase and an aqueous phase, usually octanol and water, respectively. Accordingly, in some embodiments, the substituted 3-(1-methylpyrrolidin-2-yl)pyridine of Formula I or II has a calculated or experimental log P of about 1 or greater, where log P is the log₁₀ of the partitioning coefficient of the substituted 3-(1-methylpyrrolidin-2-yl)pyridine between octanol and water. Log P values may be measured experimentally according to protocols well known to one of skill in the art. Alternatively, log P values may be calculated using commercially available software. In some embodiments, the substituted 3-(1-methylpyrrolidin-2-yl)pyridine of Formula I or II has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula I or II has a calculated log P from about 1.2 to about 1.7. In some embodiments, the substituted 3-(1-methylpyrrolidin-2-yl)pyridine having a calculated log P from about 1.2 to about 1.7 bears one or more lipophilic substituents on the pyridine ring, such as C₁-C₆ alkyl, or C₁-C₃ alkyl. In some embodiments, the lipophilic substituent is methyl, and is present at the 2, 4, 5, or 6 position of the pyridine ring. In some embodiments, the lipophilic substituted 3-(1-methylpyrrolidin-2-yl)pyridine is 2-methyl-5-(1-methylpyrrolidin-2-yl)pyridine.

The substituted 3-(1-methylpyrrolidin-2-yl)pyridine (e.g., 2-methyl-5-(1-methylpyrrolidin-2-yl)pyridine) may be present as a single enantiomer or as a mixture of enantiomers. In some embodiments, the substituted 3-(1-methylpyrrolidin-2-yl)pyridine is present in racemic form, meaning there are equal amounts of (R)- and (S)-enantiomers present. In some embodiments, the composition comprises unequal amounts of (R)- and (S)-enantiomer (i.e., is enriched in either the (R)- or (S)-enantiomer. In some embodiments, the composition predominantly comprises the substituted 3-(1-methylpyrrolidin-2-yl)pyridine (e.g., 2-methyl-5-(1-methylpyrrolidin-2-yl)pyridine) in the (R)-configuration, for example, about 90% or more of the total quantity of substituted 3-(1-methylpyrrolidin-2-yl)pyridine present is in the (R)-configuration. In some embodiments, the aerosol precursor composition predominantly comprises the substituted 3-(1-methylpyrrolidin-2-yl)pyridine (e.g., 2-methyl-5-(1-methylpyrrolidin-2-yl)pyridine) in the (S)-configuration, for example, about 90% or more of the total quantity of substituted 3-(1-methylpyrrolidin-2-yl)pyridine present is in the (S)-configuration. In some embodiments, the aerosol precursor composition comprises 95% or more of the (S)-configuration of the substituted 3-(1-methylpyrrolidin-2-yl)pyridine (e.g., 2-methyl-5-(1-methylpyrrolidin-2-yl)pyridine), based on the total amount of substituted 3-(1-methylpyrrolidin-2-yl)pyridine present.

The quantity of substituted 3-(1-methylpyrrolidin-2-yl)pyridine (e.g., 2-methyl-5-(1-methylpyrrolidin-2-yl)pyridine) present in the aerosol precursor composition may vary. Typically, the substituted 3-(1-methylpyrrolidin-2-yl)pyridine (e.g., 2-methyl-5-(1-methylpyrrolidin-2-yl)pyridine, calculated as the free base) is present in a concentration of at least about 0.001% by weight of the composition, such as in a range from about 0.01% to about 10%. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the composition. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 3% by weight, such as, e.g., from about 0.1% w/w to about 2.5%, from about 0.1% to about 2.0%, from about 0.1% to about 1.5%, from about 0.1% to about 1%, from about 0.1 to 0.75%, or from about 0.1 to about 0.5%, calculated as the free base and based on the total weight of the composition. In some embodiments, the aerosol precursor composition comprises 2-methyl-5-(1-methylpyrrolidin-2-yl)pyridine in an amount from about 0.01 to about 1% by weight, such as from about 0.1 to about 0.75% by weight, based on the total weight of the composition.

The substituted 3-(1-methylpyrrolidin-2-yl)pyridine (e.g., 2-methyl-5-(1-methylpyrrolidin-2-yl)pyridine) may be present in the aerosol precursor composition as the free base, as a salt with a suitable acid, in the form of an ion pair with an organic acid, or a combination thereof. Each of these forms is described further herein below.

### 3-(azetidin-2-yl)pyridines and 3-(azetidin-2-ylmethoxy)pyridines

In some embodiments, a composition of the disclosure comprises an optionally substituted 3-(azetidin-2-yl)pyridine or an optionally substituted 3-(azetidin-2-ylmethoxy)pyridine. As used herein, the term “substituted 3-(azetidin-2-yl)pyridine” refers to a compound having a 3-(azetidin-2-yl)pyridine scaffold and bearing one or more non-hydrogen substituents on the azetidine ring, and optionally on the pyridine ring. As used herein, the term “substituted 3-(azetidin-2-ylmethoxy)pyridine” refers to a compound having a 3-(azetidin-2-ylmethoxy)pyridine scaffold and bearing one or more non-hydrogen substituents on the azetidine ring, and optionally on the pyridine ring.

As described above, compositions of the disclosure do not contain nicotine and do not contain any compounds obtained by chemical reactions utilizing nicotine as a starting material.

In some embodiments, the 3-(azetidin-2-yl)pyridine or 3-(azetidin-2-ylmethoxy)pyridine has a structure according to Formula III:

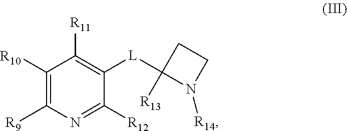

wherein:
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

In some embodiments, L is a bond.

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R³ is CH₃ and R¹⁴ is H.

In some embodiments, the 3-(azetidin-2-yl)pyridine is 3-(azetidin-2-yl)pyridine, and has a structure:

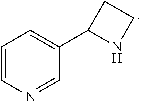

The compound 3-(azetidin-2-yl)pyridine is known in the literature. The synthesis of this compound has been reported in JOC 1979, 44(18), 3136; Med Chem Res (1993) 2:552-5633; in International Patent Application Publication No. WO2012/031220, and in U.S. Pat. Nos. 4,163,855 and 4,163,856, all of which are incorporated herein in their entireties.

In some embodiments, the 3-(azetidin-2-yl) pyridine is 3-(1-methylazetidin-2-yl)pyridine, having the structure:

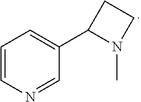

The compound 3-(1-methylazetidin-2-yl)pyridine is known in the literature. The synthesis of this compound has been reported in International Patent Application Publication No. WO2012/031220, previously incorporated by reference herein.

In some embodiments, the 3-(azetidin-2-yl) pyridine has a structure selected from the group consisting of:

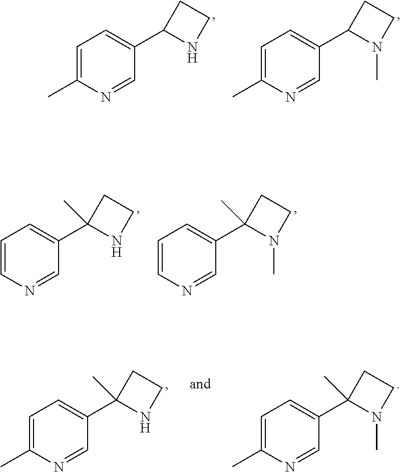

Such compounds are either known, or may be readily prepared according to adaptations of methods utilized for preparation of related 3-(azetidin-2-yl) pyridines and 3-(1-methylpyrrolidin-2-yl)pyridines described herein above. See, e.g., U.S. Pat. No. 4,163,855, previously incorporated by reference herein. The compound 5-(2-azetidinyl)-2-methylpyridine is known in the literature and has a Chemical Abstracts Registry (CAS) Number of 1270467-65-6, and the R- and S-enantiomers have CAS numbers 1213081-15-2 and 1212969-96-4, respectively.

In some embodiments, the composition comprises a 3-(azetidin-2-ylmethoxy)pyridine (i.e., L is —OCH₂—*).

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R is H.

In some embodiments, the 3-(azetidin-2-ylmethoxy)pyridine has a structure selected from the group consisting of:

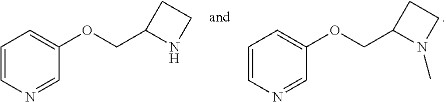

These compounds are known in the literature. The synthesis of these compounds has been reported in International Patent Application Publication No. WO2012/031220, previously incorporated by reference herein.

In some embodiments, the 3-(azetidin-2-ylmethoxy)pyridine has a structure selected from the group consisting of:

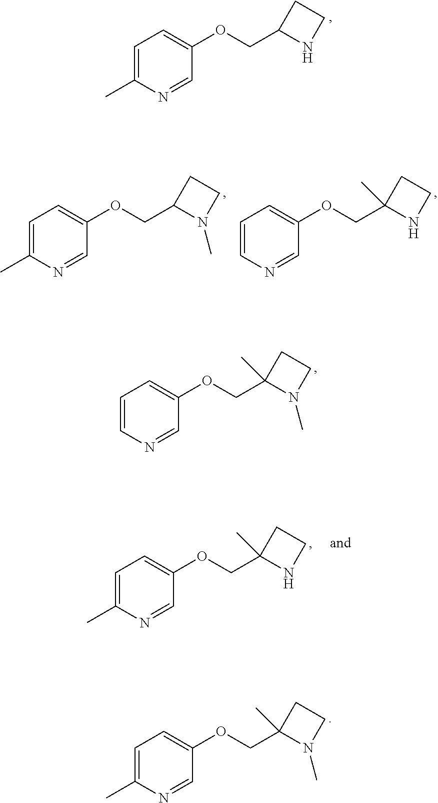

Such compounds are either known, or may be readily prepared according to adaptations of methods utilized for preparation of related the 3-(azetidin-2-ylmethoxy)pyridine, and/or the 3-(azetidin-2-yl)pyridines and substituted 3-(1-methylpyrrolidin-2-yl)pyridines described herein above.

An optionally substituted 3-(azetidin-2-yl)pyridine or optionally substituted 3-(azetidin-2-ylmethoxy)pyridine as described herein may be present as a single enantiomer or as a mixture of enantiomers. In some embodiments, the optionally substituted 3-(azetidin-2-yl)pyridine or optionally substituted 3-(azetidin-2-ylmethoxy)pyridine is present in racemic form, meaning there are equal amounts of (R)- and (S)-enantiomers present. In some embodiments, the composition comprises unequal amounts of (R)- and (S)-enantiomer (i.e., is enriched in either the (R)- or (S)-enantiomer). In some embodiments, the composition predominantly comprises the optionally substituted 3-(azetidin-2-yl)pyridine or optionally substituted 3-(azetidin-2-ylmethoxy)pyridine in the (R)-configuration, for example, about 90% or more of the total quantity of optionally substituted 3-(azetidin-2-yl)pyridine or optionally substituted 3-(azetidin-2-ylmethoxy)pyridine present is in the (R)-configuration. In some embodiments, the composition predominantly comprises the optionally substituted 3-(azetidin-2-yl)pyridine or optionally substituted 3-(azetidin-2-ylmethoxy)pyridine in the (S)-configuration, for example, about 90% or more of the total quantity of optionally substituted 3-(azetidin-2-yl)pyridine or optionally substituted 3-(azetidin-2-ylmethoxy)pyridine present is in the (S)-configuration. In some embodiments, the composition comprises 95% or more of the (S)-configuration of the optionally substituted 3-(azetidin-2-yl)pyridine or optionally substituted 3-(azetidin-2-ylmethoxy)pyridine, based on the total amount of optionally substituted 3-(azetidin-2-yl)pyridine or optionally substituted 3-(azetidin-2-ylmethoxy)pyridine present.

In some embodiments, the optionally substituted 3-(azetidin-2-yl)pyridine is non-racemic, and has one of the following structures:

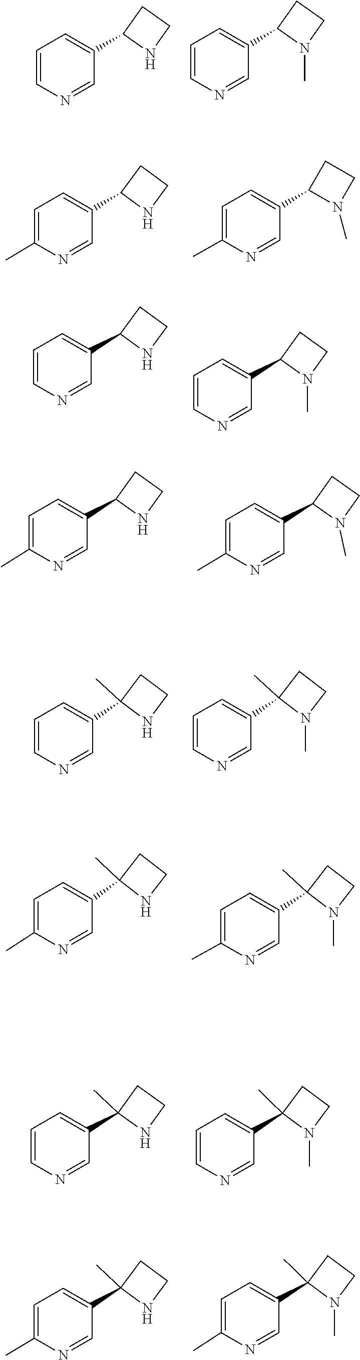

In some embodiments, the optionally substituted 3-(azetidin-2-ylmethoxy)pyridine is non-racemic, and has one of the following structures:

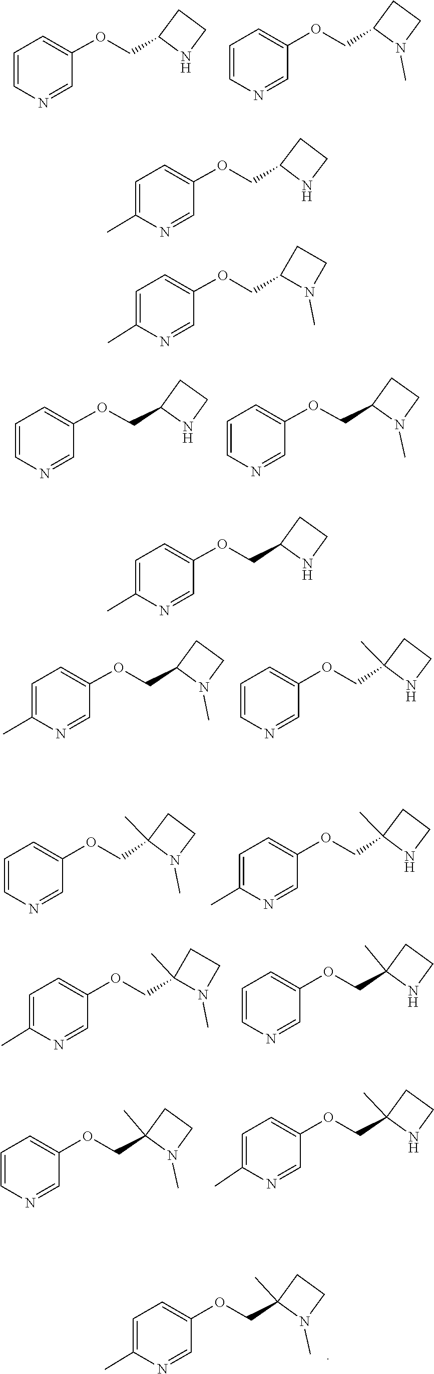

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of certain 3-(azetidin-2-yl)pyridines and 3-(azetidin-2-ylmethoxy)pyridines has been previously disclosed, for example, in the references cited herein with respect to synthesis of such compounds. Generally, these compounds exhibit high affinity for one or more subtypes of nicotinic acetylcholine receptors, particularly the a4b2 subtype. The overall pharmacological profiles have been shown to be or are expected to be comparable to that of nicotine.

The quantity of optionally substituted 3-(azetidin-2-yl)pyridine or 3-(azetidin-2-ylmethoxy)pyridine present in the composition may vary. Typically, the optionally substituted 3-(azetidin-2-yl)pyridine or 3-(azetidin-2-ylmethoxy)pyridine, calculated as the free base, is present in a concentration of at least about 0.001% by weight of the composition, such as in a range from about 0.01% to about 10%. In some embodiments, the optionally substituted 3-(azetidin-2-yl)pyridine or 3-(azetidin-2-ylmethoxy)pyridine is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the composition. In some embodiments, the optionally substituted 3-(azetidin-2-yl)pyridine or 3-(azetidin-2-ylmethoxy)pyridine is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the composition. One of skill in the art will recognize that the amount of any particular optionally substituted 3-(azetidin-2-yl)pyridine or 3-(azetidin-2-ylmethoxy)pyridine present in the composition may vary based on the potency of the compound, the composition matrix, and the desired physiological effect for the composition.

In some embodiments, the amount of optionally substituted 3-(azetidin-2-yl)pyridine or 3-(azetidin-2-ylmethoxy)pyridine in the composition is determined by potency relative to nicotine. For example, in some embodiments, the amount of optionally substituted 3-(azetidin-2-yl)pyridine or 3-(azetidin-2-ylmethoxy)pyridine is based on the potency factor as described above for substituted 3-(1-methylpyrrolidin-2-yl)pyridines. In some embodiments, an optionally substituted 3-(azetidin-2-yl)pyridine or 3-(azetidin-2-ylmethoxy)pyridine of the disclosure has a potency factor from about 0.1 to about 30, such as from about 2 to about 30.

In some embodiments, the amount of optionally substituted 3-(azetidin-2-yl)pyridine or 3-(azetidin-2-ylmethoxy)pyridine in the composition is 1 nicotine equivalent. Accordingly, in some embodiments, 1 nicotine equivalent of optionally substituted 3-(azetidin-2-yl)pyridine or 3-(azetidin-2-ylmethoxy)pyridine is an amount by weight from about 10 to about 0.03 times that of nicotine. In some embodiments, 1 nicotine equivalent of optionally substituted 3-(azetidin-2-yl)pyridine or 3-(azetidin-2-ylmethoxy)pyridine is an amount by weight from about 0.5 to about 0.03 times that of nicotine.

For example, a product comprising 2 mg of nicotine, when the nicotine is replaced by an optionally substituted 3-(azetidin-2-yl)pyridine or 3-(azetidin-2-ylmethoxy)pyridine of the disclosure, may include from about 0.06 mg to about 1 mg of the optionally substituted 3-(azetidin-2-yl)pyridine or 3-(azetidin-2-ylmethoxy)pyridine. Similarly, a product comprising 20 mg of nicotine, when the nicotine is replaced by an optionally substituted 3-(azetidin-2-yl)pyridine or 3-(azetidin-2-ylmethoxy)pyridine, may contain from about 0.6 mg to about 100 mg of the optionally substituted 3-(azetidin-2-yl)pyridine or 3-(azetidin-2-ylmethoxy)pyridine.

In some embodiments, the optionally substituted 3-(azetidin-2-yl)pyridine has the structure:

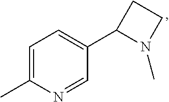

having a potency factor of about 30, meaning that in some embodiments, the amount of optionally substituted 3-(azetidin-2-yl)pyridine present may be about 3% of the amount of nicotine required to achieve the same effect.

In some embodiments, the optionally substituted 3-(azetidin-2-ylmethoxy)pyridine has the structure:

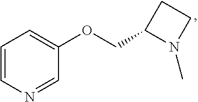

having a potency factor of about 3, meaning that in some embodiments, the amount of 3-(azetidin-2-ylmethoxy)pyridine present may be about 33% of the amount of nicotine required to achieve the same effect.

The optionally substituted 3-(azetidin-2-yl)pyridine or 3-(azetidin-2-ylmethoxy)pyridine may be present in the composition as the free base, as a salt with a suitable acid, or in the form of an ion pair with an organic acid. Each of these forms is described further herein below.

### Other Active Agents

In some embodiments, the substituted 3-(1-methylpyrrolidin-2-yl)pyridine, optionally substituted 3-(azetidin-2-yl)pyridine, or optionally 3-(azetidin-2-ylmethoxy)pyridine of the present disclosure is replaced with, or combined with, other active agents that provide the same general pharmacological profile and/or physiological effects of nicotine. Certain of these active agents may be equipotent or even more potent than nicotine with respect to binding affinity to the nicotinic acetylcholine receptor, and are expected to preserve the pharmacological effects of nicotine in vivo. Without wishing to be bound by any particular theory, in some embodiments, these compounds are believed to provide the general pharmacological profile and physiological effects of nicotine while offering the potential for one or more of greater potency, reduced product consumption, more rapid and/or complete absorption, and the like.

Example active agents of this type include, without limitation, cytisine, varenicline, acetylcholine, choline, epibatidine, lobeline, analogs thereof, or combinations thereof. Suitable analogs include any of the above-noted compounds having one or more substituents on any of the carbon atoms thereof, with example substituents including alkyl (e.g., C₁-C₃ alkyl), alkoxy, cycloalkyl, alkenyl, alkynyl, aryl, alkylaryl, amino, halogen, and cyano.

In some embodiments, the other active agent is cytisine or an analog thereof. Cytisine is a naturally occurring alkaloid present in certain plant genera, such as Laburnum and Cytisus of the family Fabaceae. Cytisine (CAS Registry No. 485-35-8) has the structure:

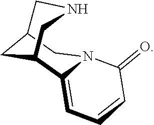

Cytisine is commercially available and has been utilized in post-Soviet states for more than 40 years as an aid to smoking cessation under the brand name Tabex (Sopharma AD). Cytisine is a partial agonist of the a4b2 nicotinic acetylcholine receptor.

In some embodiments, the other active agent is varenicline or an analog thereof. Varenicline is commercially available as Chantix® (Pfizer) and is a medication used as an aid for smoking cessation. Varenicline (CAS Registry No. 249296-44-4) has the structure:

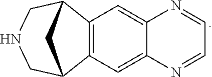 [SMILES: c1cnc2cc3c(cc2n1)[C@@H]1CNC[C@H]3C1]

Like cytisine, varenicline is a partial agonist of the a4b2 nicotinic acetylcholine receptor.

The quantity of the other active agent present in the composition may vary. Typically, the other active agent, calculated as the free base, is present in a concentration of at least about 0.001% by weight of the composition, such as in a range from about 0.01% to about 10%. In some embodiments, the other active agent is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the composition. In some embodiments, the other active agent is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the composition. One of skill in the art will recognize that the amount of any particular other active agent present in the composition may vary based on the potency of the compound, the composition matrix, and the desired physiological effect for the composition.

The other active agent may be present in the composition as the free base, as a salt with a suitable acid, or in the form of an ion pair with an organic acid. Each of these forms is described further herein below.

### Free Base

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent exhibits sufficient stability, aqueous solubility, and oral bioavailability such that the free base is suitable for inclusion in the composition. Accordingly, in some embodiments, the substituted 3-(1-methylpyrrolidin-2-yl)pyridine, optionally substituted 3-(azetidin-2-yl)pyridine, optionally 3-(azetidin-2-ylmethoxy)pyridine or other active agent is present substantially or completely as the free base. In such embodiments, one of skill in the art will recognize that the composition is substantially free of acidic components. By “substantially free” it is meant that no acidic component (e.g., inorganic acid, organic acid, or acids capable of salt, ion pair, or co-crystal formation) has been intentionally added, beyond trace amounts that may be present e.g., as an impurity in another component, or small amounts which may be present in certain flavor packages. For example, some embodiments can be characterized as having less than 0.01% by weight of any acid component, or less than 0.001%, or even 0% by weight of any acid component, based on the total weight of the composition. In some embodiments, the composition is completely free of any acid component (i.e., characterized as 0% or as having an amount below the limit of detection). In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the free base form and is adsorbed in a carrier such as a microcrystalline cellulose material to form an adsorption complex.

### Salt

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent can be employed in the form of a salt. A “salt” of such compounds is a form characterized by interaction between the said compound in ionic form and a coformer in ionic form (e.g., an acid) via the transfer of one or more protons from the coformer donor to the compound acceptor. The structure of substituted 3-(1-methylpyrrolidin-2-yl)pyridines, optionally substituted 3-(azetidin-2-yl)pyridines, and optionally 3-(azetidin-2-ylmethoxy)pyridines as disclosed herein are such that they comprise two nitrogen atoms that are capable of accepting protons from a coformer and, accordingly, can be present in non-protonated, mono-protonated, and/or di-protonated form in a given sample. Salts of substituted 3-(1-methylpyrrolidin-2-yl)pyridine, optionally substituted 3-(azetidin-2-yl)pyridine, optionally 3-(azetidin-2-ylmethoxy)pyridine, or other active agent can be provided using the types of ingredients and techniques set forth for nicotine in U.S. Pat. No. 2,033,909 to Cox et al. and Perfetti, Beitrage Tabakforschung Int., 12: 43-54 (1983), which are incorporated herein by reference. Suitable salts are generally water soluble. Suitable acids for formation of salts (mono- and di-) include, but are not limited to, acetic acid, adipic acid, ascorbic acid, capric acid, citric acid, D-glucuronic acid, D-gluconic acid, lactic acid, galactaric acid, hippuric acid, hydrochloric acid, L-aspartic acid, L-glutamic acid, L-glutaric acid, glycerophosphoric acid, glycolic acid, lauric acid, DL-malic acid, L-malic acid; tartaric acid, palmitic acid, phosphoric acid, sebacic acid, stearic acid, succinic acid, sulfuric acid, thiocyanic acid, (+)-camphoric acid, 1,5-naphthalenedisulfonic acid, 1-hydroxy-2-naphthoic, 2,5-dihydroxybenzoic acid, benzenesulfonic acid, benzoic acid, caprylic acid, cyclamic acid, ethanesulfonic acid, fumaric acid, D-glucoheptonic acid, 4-hydroxybenzoic acid, isobutyric acid, ketoglutaric acid, 2-ketobutyric acid, lactobionic acid, maleic acid, malonic acid, methanesulfonic acid, naphthalene-2-sulfonic acid, nicotinic acid, oleic acid, orotic acid, oxalic acid, pamoic acid, pivalic acid, propionic acid, L-pyroglutamic acid, p-toluenesulfonic acid, (1S)-camphor-10-sulfonic acid, 4-acetamidobenzoic acid, 4-aminosalicylic acid, N-acetyl-4-aminosalicylic acid, caproic acid, dichloroacetic acid, hydrobromic acid, DL-mandelic acid, L-mandelic acid, nitric acid, formic acid, salicylic acid, cinnamic acid, undecylenic acid, isothionic acid, lauric acid, 2-hydroxybenzoic acid, trans-2-hexanoic acid, trimesic acid, 5-nitroisophthalic acid and zinc chloride monohydrate (forming a hydrated zinc chloride complex salt). In some embodiments, the salt is with an organic acid as described herein below.

In some embodiments, a hydrophilic acid is chosen so as to increase water solubility and/or decrease lipophilicity of the salt. Lipophilicity of a salt of a compound as disclosed herein can also be expressed as log D, which is the logarithm of the distribution coefficient, a measure of the pH-dependent differential solubility between an octanol phase and an aqueous phase of all species (ionized and un-ionized) in an octanol/aqueous system, represented by the formula:

$$log {D}_{oct / wat} = log ( \frac{{[ solute ]}_{octanol}}{{[ solute ]}_{water}^{ionized} + {[ solute ]}_{water}^{neutral}} ) .$$

Log D is a commonly used descriptor for the lipophilicity of ionizable compounds. Log D values can be calculated using commercial software or may be determined experimentally in a similar manner to log P but instead of using water, the aqueous phase is adjusted to a specific pH using a buffer. Log D is pH dependent and therefore requires that the pH at which the log D was measured be specified.

When the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of a salt, it is generally preferred that the salt have a relatively low log D, indicative of good water solubility. Without wishing to be bound by theory, it is believed that highly water-soluble salt forms may exhibit a high rate of dissolution, which may be favorable in certain embodiments. Accordingly, in some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent salt has a log D from about −1.0 to about 3 at a pH in a range from about 3 to about 11, such as from about −0.5 to about 2, about −0.3 to about 1, or about −0.1 to about 0.

In some embodiments, the selection of acid used to make a salt is performed on the basis of sensory effects of the salt, such as taste. Surprisingly, according to the present disclosure, it has been found that salts of certain organic acids, such as galactaric acid, offer a better taste sensation relative to salts of acids such as tartaric or phthalic acids.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of a salt with tartaric acid, succinic acid, orotic acid, fumaric acid, pyroglutamic acid, or galactaric acid. In some embodiments, the substituted 3-(1-methylpyrrolidin-2-yl)pyridine, optionally substituted 3-(azetidin-2-yl)pyridine, or optionally 3-(azetidin-2-ylmethoxy)pyridine is present in the form of a salt with succinic acid or galactaric acid.

The stoichiometry of the salts as described herein can vary. For example, in some embodiments, the stoichiometry can range from about 5:1 to about 1:5 molar equivalents of compound:acid, including any whole or fractional value in between. In some embodiments, the molar ratio of compound to acid is about 2:1, about 1:1, or about 1:2. In some embodiments, the compound is present in the form of a bitartrate salt. Hydrates and other solvates of salts are further contemplated herein.

The salts as described herein can, in some embodiments, exist in various polymorphic and pseudopolymorphic forms. Polymorphism is the ability of a crystalline material to exist in more than one form or crystal structure. Polymorphism can result, e.g., from the existence of different crystal packing structures (packing polymorphism) or from the existence of different conformers of the same molecule (conformational polymorphism). Pseudopolymorphism is the result of hydration or solvation of a material and is also referred to as solvomorphism.

### Organic Acid

In some embodiments, the composition comprises an organic acid. In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2-(4-isobutylphenyl)propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid. Examples of suitable acids include, but are not limited to, the list of organic acids in Table 1.

TABLE 1
Non-limiting examples of suitable organic acids
Acid Name

benzoic acid
phenylacetic
p-toluic acid
ethyl benzoic acid
isopropyl benzoic acid
4-phenylbutyric
2-(4-Isobutylphenyl)propanoic acid
2-napthoxyacetic acid
napthylacetic acid
heptanoic acid
octanoic acid
nonanoic acid
decanoic acid
9-deceneoic acid
2-deceneoic acid
10-undecenoic acid
dodecandioic acid
dodecanoic acid
myristic acid
palmitic acid
stearic acid
cyclohexanebutanoic acid
1-heptanesulfonic acid
1-octanesulfonic acid
1-nonanesulfonic acid
monooctyl succinate
tocopherol succinate
norbixin ((2E,4E,6E,8E,10E,12E,14E,16E,18E)-4,8,13,17-
tetramethylicosa-2,4,6,8,10,12,14,16,18-nonaenedioic acid)
bixin ((2E,4E,6E,8E,10E,12E,14E,16Z,18E)-20-methoxy-
4,8,13,17-tetramethyl-20-oxoicosa-2,4,6,8,10,12,14,16,18-
nonaenoic acid)

The selection of organic acid may depend on certain properties. For example, an organic acid should be one recognized as safe for human consumption, and which has acceptable flavor, odor, volatility, stability, and the like. Determination of appropriate organic acids is within the purview of one of skill in the art.

In some embodiments, more than one organic acid may be present. For example, the composition may comprise two, or three, or four, or more organic acids. Accordingly, reference herein to “an organic acid” contemplates mixtures of two or more organic acids. The relative amounts of the multiple organic acids may vary. For example, a composition may comprise equal amounts of two, or three, or more organic acids, or may comprise different relative amounts. Without wishing to be bound by theory, it is believed that a combination of different organic acids may provide a concentration of any single organic acid in the composition which remains below the threshold which would be found objectionable from a sensory perspective.

The amount of organic acid present in the composition, relative to the substituted 3-(1-methylpyrrolidin-2-yl)pyridine (e.g., 2-methyl-5-(1-methylpyrrolidin-2-yl)pyridine), may vary. In some embodiments, the composition comprises from about 0.05, about 0.1, about 1, about 1.5, about 2, or about 5, to about 10, about 15, or about 20 molar equivalents of the organic acid relative to the substituted 3-(1-methylpyrrolidin-2-yl)pyridine, calculated as the free base of the substituted 3-(1-methylpyrrolidin-2-yl)pyridine.

In some embodiments, the composition comprises from about 2 to about 10, or from about 2 to about 5 molar equivalents of the organic acid relative to the substituted 3-(1-methylpyrrolidin-2-yl)pyridine, on a free-base basis. In some embodiments, the organic acid is present in a molar ratio with the substituted 3-(1-methylpyrrolidin-2-yl)pyridine from about 2, about 3, about 4, or about 5, to about 6, about 7, about 8, about 9, or about 10. In embodiments wherein more than one organic acid it is to be understood that such molar ratios reflect the totality of the organic acids present.

In some embodiments, the organic acid and the substituted 3-(1-methylpyrrolidin-2-yl)pyridine are present in a molar ratio in a range from about 1:10 to about 2:1, such as about 1:2 to about 2:1, or about 0.9:1 to about 1.1:1, such as wherein the molar ratio is 1:10 to about 1:1 or about 1:10 to about 1:1.1 or about 1:5 to about 1:1.2 or about 1:4 to about 1:1.5. In some embodiments, the organic acid is used in an amount of no more than equimolar (1:1). In some embodiments, the organic acid is used in an amount of less than equimolar, such as a molar ratio of organic acid to substituted 3-(1-methylpyrrolidin-2-yl)pyridine of about 1:10 to about 1:1.1 or about 1:5 to about 1:1.2 or about 1:4 to about 1:1.5. In some embodiments, use of less than equimolar amounts of organic acid can improve the sensory characteristics of aerosol produced using the aerosolizable composition.

In some embodiments, the aerosol precursor composition comprises one or more of the organic acids described herein above. The substituted 3-(1-methylpyrrolidin-2-yl)pyridine and the one or more organic acids may be present independently, in the form of a salt, or as a cocrystal. In some embodiments, the aerosol precursor composition comprises one or more of lactic acid, levulinic acid, benzoic acid, succinic acid, galactaric acid, orotic acid, fumaric acid, pyroglutamic acid, and tartaric acid, and at least a portion of said acid is present in the form of a salt with the substituted 3-(1-methylpyrrolidin-2-yl)pyridine.

In some embodiments, the aerosol precursor composition comprises a lactic acid salt of the substituted 3-(1-methylpyrrolidin-2-yl)pyridine.

In some embodiments, the aerosol precursor composition comprises a levulinic acid salt of the substituted 3-(1-methylpyrrolidin-2-yl)pyridine.

In some embodiments, the aerosol precursor composition comprises a galactaric acid salt of the substituted 3-(1-methylpyrrolidin-2-yl)pyridine.

In some embodiments, the aerosol precursor composition comprises a benzoic acid salt of the substituted 3-(1-methylpyrrolidin-2-yl)pyridine.

The stoichiometry of such lactate, levulinate, tartrate, galactarate, or benzoate salts may vary. Accordingly, the molar ratio of lactic, levulinic, tartaric, or galactaric acid to substituted 3-(1-methylpyrrolidin-2-yl)pyridine may be in a range from about 3:1 to about 1:3, including any whole or fractional value in between, such as for example about 2:1, about 1.5:1, about 1:1, about 1:1.5, or about 1:2. In some embodiments, these organic acids are used in an amount of less than equimolar, such as a molar ratio of organic acid to substituted 3-(1-methylpyrrolidin-2-yl)pyridine of about 1:10 to about 1:1.1 or about 1:5 to about 1:1.2 or about 1:4 to about 1:1.5. In some embodiments, use of less than equimolar amounts of these organic acids can improve the sensory characteristics of aerosol produced using the aerosolizable composition.

In some embodiments, the composition may be characterized as substantially or completely free of organic acids (i.e., having less than 0.01% by weight of organic acid, or less than 0.001%, or even 0% by weight of organic acid, based on the total weight of the composition, or as having an amount of organic acid below the limit of detection).

In some embodiments, the aerosol precursor composition comprises one or more further components, such as active agent, a flavorant, or other functional materials. Each of these further components is further described herein below.

### Resin Complex

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent may be present in the form of a polymer complex, where the compound is bound to an acidic polymer. The polymer of such a complex can be any polymer (including homopolymers or all types of copolymers) with acidic functionalities, e.g., a polymeric cation exchange resin. In some embodiments, the polymer comprises acidic sites that can be classified as strongly acidic, weakly acidic, or of intermediate acidity (depending, e.g., on the strength of the acid from which they are derived). In some embodiments, the polymer comprises weakly acidic sites and can be referred to as a weakly acidic cation exchange resin. Non-limiting examples of acidic sites include, e.g., carboxylic acids, sulfonic acids, phosphonous acids, phosphonic acids, phosphoric acids, iminodiacetic acids, and phenolic groups (e.g., as disclosed in Adams et al., J. Soc. Chem. Ind. 54, IT (1935), which is incorporated herein by reference). Suitable polymers include, but are not limited to, addition polymers of styrene and divinylbenzene, divinylbenzene and methacrylic acid, divinylbenzene and acrylic acid, phenolic resins, or cellulose, dextran or pectin cross-linked with, e.g., epichlorohydrin. In some embodiments, the polymer comprises cross-linked moieties. Various acidic ion-exchange resins which are known in the art and are suitable for formation of complexes, include, but are not limited to, polymethacrylic acid resins such as DuPont™ Amberlite™ IRP64, DuPont™ Amberlite™ IRP69, Purolite™ C115HMR, Doshion™ P551, and polyacrylic carbomers, such as Carbopol 974P. See, for example, U.S. Pat. No. 3,901,248 to Lichtneckert et al., which is incorporated herein by reference. In some embodiments, when the substituted 3-(1-methylpyrrolidin-2-yl)pyridine, optionally substituted 3-(azetidin-2-yl)pyridine, or optionally 3-(azetidin-2-ylmethoxy)pyridine is present in the form of a polymer complex, the composition further comprises a divalent metal buffer, such as a calcium or magnesium salt (e.g., carbonate, bicarbonate, oxide, acetate, or the like).

### Cocrystal

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent may be present in the form of a co-crystal with at least one other component (“coformer”), both in neutral form. Specifically, as defined in a US FDA industry guidance document, a co-crystal is a solid that is a crystalline material composed of two or more molecules in the same crystal lattice, where the components are in a neutral state and interact via nonionic interactions. See U.S. Department of Health and Human Services, Food and Drug Administration, Guidance for Industry: Regulatory Classification of Pharmaceutical Co-Crystals (April 2013), which is incorporated herein by reference. This form is different and distinct from both salts and ion pairs, each described herein. Specifically, co-crystals can generally be distinguished from salts (and ion pairs) by the absence of a proton transfer between the components (i.e., a substituted 3-(1-methylpyrrolidin-2-yl)pyridine and the one or more coformers) in a co-crystal. The crystalline structure of the co-crystal is generally held together by freely reversible, non-covalent interactions. Co-crystals typically comprise the substituted 3-(1-methylpyrrolidin-2-yl)pyridine, optionally substituted 3-(azetidin-2-yl)pyridine, or optionally 3-(azetidin-2-ylmethoxy)pyridine and coformer in a defined stoichiometric ratio. In some embodiments, co-crystals can encompass hydrates, solvates, and clathrates. Co-crystals can comprise the substituted 3-(1-methylpyrrolidin-2-yl)pyridine, optionally substituted 3-(azetidin-2-yl)pyridine, optionally 3-(azetidin-2-ylmethoxy)pyridine, or other active agent in combination with an organic and/or an inorganic coformer.

Examples of suitable coformers include, but are not limited to, acetamidobenzoic acid, L-proline, tromethamine, urea, xylitol, caffeine, glycine/glycine anhydride, vanillin, methyl 4-hydroxybenzoate(methylparaben), succinimide, L-alanine, mannitol, L-phenylalanine, saccharin, propylparaben, N-methylglucamine, L-tyrosine, gentisic acid, sorbic acid, benzoic acid, L-methionine, maltol, L-lysine, tromethamine, nicotinamide, isonicotinamide, phenylalanine, benzoquinone, terephthalaldehyde, 4-hydroxybenzoic acid, pyruvic acid, 1-hydroxy-2-naphthoic acid, 4-aminobenzoic acid, vanillic acid, ethyl vanillin, isonicotinic acid, gallic acid, menthol (e.g., racemic menthol or (−)-menthol), paracetamol, aspirin, ibuprofen, naproxen, ketoprofen, flurbiprofen, glucose, serine, malic acid, acetamide, sulfacetamide, benzoic acid, creatine, 2-hydroxyethanesulfonic acid, clofibric acid, taurine (tauric acid), iproniazid, L-histadine, L-arginine, L-asparagine, glutamine, L-cysteine, alanine, valine, isoleucine, leucine, morpholine, theronine, N-methylglucamine, 3-hydroxy-2-oxopropionic acid; 2-oxobutyric acid (2-ketobutyric acid), 3-methyl-2-oxobutanoic acid; 3-methyl-2-oxopentanoic acid; 4-methyl-2-oxopentanoic acid; and 2-oxopentanedioic acid, 2-oxo-3-phenylpropionic acid; 5-oxooctanoic acid; and 5-oxodecanoic acid, aldonic acids (e.g., glyceric acid, xylonic acid, gluconic acid, and ascorbic acid), ulosonic acids (e.g., neuraminic acid and ketodeoxyoctulosonic acid), uronic acids (e.g., glucuronic acid, galacturonic acid, and iduronic acid), aldaric acids (e.g., tartaric acid, meso-galactaric acid/mucic acid, and D-glucaric acid/saccharic acid), galactaric acid), and polyfunctional aromatic acids.

In some embodiments, the conformer is a polyfunctional aromatic acid. Polyfunctional aromatic acids often comprise a substituted or unsubstituted phenyl group as the aromatic component, but can alternatively comprise another aromatic moiety, e.g., pyridine, pyrazine, imidazole, pyrazole, oxazole, thiophene, naphthalene, anthracene, and phenanthrene. Substituents on the optionally substituted aromatic acids may be any type of substituent, including, but not limited to, halo (e.g., Cl, F, Br, and I); alkyl, halogenated alkyl (e.g., CF₃, 2-Br-ethyl, CH₂F, CH₂Cl, CH₂CF₃, or CF₂CF₃); alkenyl, hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate groups. Example polyfunctional aromatic acids can be, for example:
substituted and unsubstituted aromatic dicarboxylic acids (e.g., 1,2-benzenedicarboxylic acid (phthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid;
substituted and unsubstituted hydroxybenzoic acids (e.g., 2-hydroxybenzoic acid (salicylic acid), 3-hydroxybenzoic acid, 4-hydroxybenzoic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo-4-hydroxybenzoic acid, 3-bromo-5-hydroxybenzoic acid, 4-bromo-2-hydroxybenzoic acid, 5-bromo-2-hydroxybenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-hydroxybenzoic acid, 4-fluoro-3-hydroxybenzoic acid, 2-fluoro-4-hydroxybenzoic acid, 5-fluoro-2-hydroxybenzoic acid, 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3-formyl-4-hydroxybenzoic acid, 5-(acetylamino)-2-hydroxybenzoic acid), 4-nitro-2-hydroxybenzoic acid, 3,5-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluoro-4-hydroxybenzoic acid, 3,4-difluoro-2-hydroxybenzoic acid, 3,5-dibromo-2-hydroxybenzoic acid, 3,5-diodo-2-hydroxybenzoic acid, 4-amino-5-chloro-2-hydroxybenzoic acid, 3,5-dinitro-2-hydroxybenzoic acid, 2,4,6-tribromo-2-hydroxybenzoic acid, 2,3,5,6-tetrafluoro-4-hydroxybenzoic acid, and 2,3,4,5-tetrafluoro-6-hydroxybenzoic acid);
substituted and unsubstituted dihydroxybenzoic acids (e.g., 2,3-dihydroxybenzoic acid (pyrocatechuic acid/hypogallic acid), 2,4-dihydroxybenzoic acid (3-resorcylic acid), 2,5-dihydroxybenzoic acid (gentisic acid/hydroquinonecarboxylic acid), 2,6-dihydroxybenzoic acid (7-resorcylic acid), 3,4-dihydroxybenzoic acid (protocatechuic acid), 3,5-dihydroxybenzoic acid (a-resorcylic acid), 4-hydroxy-3-methoxybenzoic acid (vanillic acid), 6-methyl-2,4-dihydroxybenzoic acid (orsellenic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, and 5-amino-3-chloro-2,4-dihydroxybenzoic acid);
substituted and unsubstituted trihydroxybenzoic acids (e.g., 2,3,4-trihydroxybenzoic acid, 2,4,5-trihydroxybenzoic acid, 2,4,6-trihydroxybenzoic acid (phloroglucinol carboxylic acid), and 3,4,5-trihydroxybenzoic acid (gallic acid));
substituted and unsubstituted aromatic tricarboxylic acids (e.g., 1,2,3-benzenetricarboxylic acid, 1,2,4-benzenetricarboxylic acid (trimellitic acid); and
substituted and unsubstituted aromatic tetracarboxylic acids (e.g., 1,2,3,4-benzenetetracarboxylic acid (mellophanic acid) and 1,2,4,5-benzenetetracarboxylic acid (pyromellitic acid). Further contemplated are various combinations of any of the foregoing acids.

In some embodiments, the coformer is L-malic acid, succinic acid, or a combination thereof. In some embodiments, the coformer is 1,1,6,6-tetraphenyl-2,4-hexidiyne-1,6-diol. In some embodiments, the coformer is di-iodotetrafluoro benzene, 4,4′-diiodooctafluorobiphenyl, or 1,4-bis(diphenylhydroxymethyl)benzene. In some embodiments, the coformer is orotic acid.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of a salt co-crystal. A “salt co-crystal” is a type of hybrid structure with both salt and co-crystal characteristics. Typically, a substituted 3-(1-methylpyrrolidin-2-yl)pyridine, optionally substituted 3-(azetidin-2-yl)pyridine, optionally 3-(azetidin-2-ylmethoxy)pyridine molecule, or other active agent within a salt co-crystal is associated with at least two coformers (which may be the same or different), wherein one coformer is in ionic form (e.g., an acid) and transfers a proton to the substituted 3-(1-methylpyrrolidin-2-yl)pyridine, optionally substituted 3-(azetidin-2-yl)pyridine, optionally 3-(azetidin-2-ylmethoxy)pyridine molecule, or other active agent, and wherein a second coformer does not transfer a proton to the substituted 3-(1-methylpyrrolidin-2-yl)pyridine, optionally substituted 3-(azetidin-2-yl)pyridine, optionally 3-(azetidin-2-ylmethoxy)pyridine molecule, or other active agent. Suitable acids and coformers are generally those described herein above with respect to salts and co-crystals.

The stoichiometry of the co-crystals and salt co-crystals described herein can vary. For example, in certain embodiments, where two components are present, the stoichiometry can range in certain embodiments from about 5:1 to about 1:5 compound:coformer. Where more than one coformer is used to form a co-crystal or salt co-crystal, the ratios of the coformers with respect to both the substituted 3-(1-methylpyrrolidin-2-yl)pyridine, optionally substituted 3-(azetidin-2-yl)pyridine, optionally 3-(azetidin-2-ylmethoxy)pyridine, or other active agent and to one another can also vary.

The co-crystals and salt co-crystals described herein can, in some embodiments, exist in various polymorphic and pseudopolymorphic forms, as well as solvates and hydrates.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of a salt-co-crystal. In some embodiments, the salt-co-crystal is a bis-orotic acid salt-co-crystal. In some embodiments, the bis-orotic acid salt-co-crystal is a hemi-hydrate.

### Ion Pairing

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of an ion pair. Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (e.g., a substituted 3-(1-methylpyrrolidin-2-yl)pyridine and the conjugate base of a suitable acid). By “conjugate base” is meant the base resulting from deprotonation of the corresponding acid (e.g., benzoate is the conjugate base of benzoic acid). In embodiments comprising ion pairing, on average, a certain population of these ion pairs exists at any given time, although the formation and dissociation of ion pairs is continuous. In some embodiments, in the composition as disclosed herein, and/or upon oral use of said composition (e.g., upon contact with saliva), the substituted 3-(1-methylpyrrolidin-2-yl)pyridine, optionally substituted 3-(azetidin-2-yl)pyridine, optionally 3-(azetidin-2-ylmethoxy)pyridine, or other active agent and the conjugate base of an acid exist at least partially in the form of an ion pair. Ion pairing is further described in, for example, International Patent Application Publication No. WO2021/050741 to Poole et al., and US Application Publication Nos. 2021/0068447 to Keller et al., 2023/0138306A1 to Zawadzki et al., and 2022/0346434 to Von Cosmos et al., each of which is incorporated herein by reference.

One of skill in the art will recognize that the extent of ion pairing in the disclosed composition, both before and during use by the consumer, may vary based on, for example, pH, the nature of the acid, the concentration of substituted 3-(1-methylpyrrolidin-2-yl)pyridine, optionally substituted 3-(azetidin-2-yl)pyridine, optionally 3-(azetidin-2-ylmethoxy)pyridine, or other active agent, the concentration of the acid or conjugate base of the acid present in the composition, the moisture content of the composition, the ionic strength of the composition, and the like. One of skill in the art will also recognize that ion pairing is an equilibrium process influenced by the foregoing variables. Accordingly, quantification of the extent of ion pairing is difficult or impossible by calculation or direct observation. However, the presence of ion pairing may be demonstrated through surrogate measures, such as partitioning of the substituted 3-(1-methylpyrrolidin-2-yl)pyridine, optionally substituted 3-(azetidin-2-yl)pyridine, optionally 3-(azetidin-2-ylmethoxy)pyridine, or other active agent between octanol and water, or by performing membrane permeation studies of aqueous solutions of, for example, the substituted 3-(1-methylpyrrolidin-2-yl)pyridine plus acids and/or their conjugate bases. An octanol-water partitioning favoring distribution of an ion pair into octanol is predictive of good absorption of the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active agent through the oral mucosa. However, as described above, in some embodiments, the properties of the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active agent are such that no ion pairing is required, and accordingly, the composition is substantially or completely free of any ion pairing. By “substantially free” it is meant that no measurable degree of ion pairing is present.

In embodiments where ion pairing is desired, the composition comprises an organic acid, an alkali metal salt of an organic acid, or both. In such embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent is associated with at least a portion of the organic acid, the alkali metal salt thereof, or a combination thereof in the form an ion pair. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient). For the avoidance of doubt, reference herein to an “organic acid” is intended to distinguish the acid present in ion paired forms over the acid which may be present in salts, co-crystal, and salt co-crystals. While one of skill in the art will recognize that certain organic acids suitable for formation of ion pairs overlap with those identified as suitable for salt or co-crystal formation, it is to be understood that the particular acid used for each of salts, co-crystals, and ion pairs are to be selected specifically for each such embodiment, and reference herein to an organic acid is specific to acids suitable for ion pairing. Accordingly, the presence in the composition of an organic acid as defined below is to be interpreted solely with respect to ion pairing, even if such organic acid is also suitable for salt formation or co-crystal formation, and the presence of such an organic acid does not imply that a salt or co-crystal is present unless explicitly identified. Further, in embodiments where there is no ion pairing intended, the composition may be characterized as substantially or completely free of organic acids (i.e., having less than 0.001% by weight of organic acid, or less than 0.0001%, or even 0% by weight of organic acid, based on the total weight of the composition, or as having an amount of organic acid below the limit of detection). This is not to be interpreted as meaning that the composition is substantially or completely free of substituted 3-(1-methylpyrrolidin-2-yl)pyridine salts or substituted 3-(1-methylpyrrolidin-2-yl)pyridine co-crystals unless explicitly recited.

The amount of organic acid or alkali metal salt thereof present in the composition, relative to the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active agent, may vary. Generally, as the concentration of the organic acid (or the conjugate base thereof) increases, the percent of substituted 3-(1-methylpyrrolidin-2-yl)pyridine, optionally substituted 3-(azetidin-2-yl)pyridine, optionally 3-(azetidin-2-ylmethoxy)pyridine, or other active agent that is ion paired with the organic acid increases. This typically increases the partitioning of the substituted 3-(1-methylpyrrolidin-2-yl)pyridine, optionally substituted 3-(azetidin-2-yl)pyridine, optionally 3-(azetidin-2-ylmethoxy)pyridine, or other active agent in the form of an ion pair, into octanol versus water as measured by the log P (the log₁₀ of the partitioning coefficient). In some embodiments, the composition comprises from about 0.05, about 0.1, about 1, about 1.5, about 2, or about 5, to about 10, about 15, or about 20 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally 3-(azetidin-2-ylmethoxy)pyridine, or other active agent, calculated as the free base of the substituted 3-(1-methylpyrrolidin-2-yl)pyridine, optionally substituted 3-(azetidin-2-yl)pyridine, optionally 3-(azetidin-2-ylmethoxy)pyridine, or other active agent.

In some embodiments, the composition comprises from about 2 to about 10, or from about 2 to about 5 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally 3-(azetidin-2-ylmethoxy)pyridine, or other active agent on a free-base basis. In some embodiments, the organic acid, the alkali metal salt thereof, or the combination thereof, is present in a molar ratio with the substituted 3-(1-methylpyrrolidin-2-yl)pyridine, optionally substituted 3-(azetidin-2-yl)pyridine, optionally 3-(azetidin-2-ylmethoxy)pyridine, or other active agent from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present. In some embodiments, the composition comprises benzoic acid and sodium benzoate wherein a total amount of benzoate (i.e., benzoic acid and benzoate) is in a molar ratio in a range from about 3 to about 5 relative to the substituted 3-(1-methylpyrrolidin-2-yl)pyridine, optionally substituted 3-(azetidin-2-yl)pyridine, optionally 3-(azetidin-2-ylmethoxy)pyridine, or other active agent. In some embodiments, the molar ratio of the total amount of benzoate to the substituted 3-(1-methylpyrrolidin-2-yl)pyridine, optionally substituted 3-(azetidin-2-yl)pyridine, optionally 3-(azetidin-2-ylmethoxy)pyridine, or other active agent is about 3.2 or about 4.8.

In some embodiments, the organic acid inclusion is sufficient to provide a composition pH of from about 4.0 to about 9.0, such as from about 4.5 to about 7.0, or from about 5.5 to about 7.0, from about 4.0 to about 5.5, or from about 7.0 to about 9.0. Reference herein to “a composition pH” means the pH of an aqueous solution of the composition prepared by dissolving or suspending 5 grams of composition in 95 grams of water and measuring the pH of the resulting solution with a calibrated pH meter.

In some embodiments, the organic acid inclusion is sufficient to provide a composition pH of from about 4.5 to about 6.5, for example, from about 4.5, about 5.0, or about 5.5, to about 6.0, or about 6.5. In some embodiments, the desired composition pH is from about 4.5 to about 6.5, and the organic acid is provided in a quantity sufficient to provide such a pH. In some embodiments, the organic acid is provided in a quantity sufficient to provide a pH of the composition of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In some embodiments, a mineral acid (e.g., hydrochloric acid, sulfuric acid, phosphoric acid, or the like), alone or in combination with an organic acid, is added to adjust the pH of the composition to the desired value. In some embodiments, a buffer (e.g., a buffer as described herein below) is added to the composition to the desired value, and/or to maintain the pH of the composition at the desired value.

In some embodiments, the oral composition further comprises a solubility enhancer to increase the solubility of one or more of the organic acid or salt thereof. Suitable solubility enhancers include, but are not limited to, humectants as described herein, such as glycerol or propylene glycol.

### Other Active Agents

In some embodiments, the aerosol precursor composition comprises an active agent in addition to the active agents described herein above. These additional active agents, include, but are not limited to, botanical extracts, cannabinoids, terpenes, tobacco extracts, and combinations thereof.

### Botanical Extract

In some embodiments, the other active agent comprises one or more botanical extracts. As used herein, the term “botanical extract” refers to an extract of any plant material or fungal-derived material, including plant materials subjected to heat treatment, fermentation, or other treatment processes capable of altering the chemical nature of the material. For the purposes of the present disclosure, a “botanical” extract includes but is not limited to extracts of “herbal materials,” which refer to seed-producing plants that do not develop persistent woody tissue and are often valued for their medicinal or sensory characteristics (e.g., teas or tisanes). Botanical extracts include extracts of either tobacco or non-tobacco materials. Reference to botanical material as “non-tobacco” is intended to exclude tobacco materials (i.e., does not include any Nicotiana species).

Non-limiting examples of botanical materials from which extracts can be prepared include without limitation acai berry, alfalfa, allspice, annatto seed, apricot oil, ashwagandha, bacopa monniera, baobab, basil, bee balm, beet root, wild bergamot, black pepper, blueberries, borage seed oil, bugleweed, cacao, calamus root, catnip, catuaba, cayenne pepper, Centella asiatica, chaga mushroom, Chai-hu, chamomile, cherry blossom, chervil, chlorophyll, cinnamon, dark chocolate, citrus, cocoa, comfrey leaf and root, gingko biloba, ginseng, goji berries, grape seed, green tea, black tea, black cohosh, cayenne, chamomile, cloves, cocoa powder, cordyceps, cranberry, curcumin, damiana, dandelion, Dorstenia arifolia, Dorstenia odorata, echinacea, eucalyptus, fennel, feverfew, Galphimia glauca, garlic, ginger, ginseng (e.g., Panax ginseng), goldenseal, green tea, grapefruit, Griffonia simplicifolia, guarana, gutu kola, hawthorn, hemp, hibiscus flower, honeybush, hops, jasmine, jiaogulan, Kaempferia parviflora (Thai ginseng), kava, lavender, lemon balm, lemongrass, licorice, Lion's mane, lutein, maca, matcha, Nardostachys chinensis, marjoram, milk thistle, mints (menthe), oolong tea, orange, oregano, papaya, pennyroyal, peppermint, potato peel, primrose, quercetin, red clover, resveratrol, Rhizoma gastrodiae, Rhodiola, rooibos, rooibos (red or green), rose essential oil, rosehip, rosemary, sage, clary sage, savory, saw palmetto, Sceletium tortuosum, Schisandra, Silybum marianum, Skullcap, spearmint, Spikenard, spirulina, slippery elm bark, sorghum bran hi-tannin, sorghum grain hi-tannin, Saint John's Wort, sumac bran, terpenes, thyme, tisanes, tobacco, turmeric, Turnera aphrodisiaca, uva ursi, valerian, Viola odorata, white mulberry, wild yam root, wintergreen, Withania somnifera, yacon root, yellow dock, yerba mate, and yerba santa.

When present, a botanical extract is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the aerosol precursor composition.

### Cannabinoids

In some embodiments, the other active agent comprises one or more cannabinoids. As used herein, the term “cannabinoid” refers to a class of diverse natural or synthetic chemical compounds that acts on cannabinoid receptors (e.g., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids).

Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

In certain embodiments, the cannabinoid is selected from tetrahydrocannabinol (THC), the primary psychoactive compound in cannabis, and cannabidiol (CBD), another major constituent of the plant, but which is devoid of psychoactivity. All of the above compounds can be used in the form of an isolate from plant material or synthetically derived. Certain cannabinoids, including but not limited to CBD and THC, may exist in more than one isomeric form, for example Δ8- and Δ9-THC. Such isomeric forms may be naturally occurring or may be synthetic. For avoidance of doubt, reference within the present disclosure to a “cannabinoid” is intended to be inclusive of any and all isomeric forms thereof.

In some embodiments, the cannabinoid comprises at least tetrahydrocannabinol (THC). In some embodiments, the cannabinoid is tetrahydrocannabinol (THC). In some embodiments, the THC is Δ8-THC. In some embodiments, the THC is Δ9-THC.

In some embodiments, the cannabinoid comprises at least cannabidiol (CBD). In some embodiments, the cannabinoid is cannabidiol (CBD). In some embodiments, the CBD is synthetic CBD. In some embodiments, the CBD is Δ8-CBD. In some embodiments, the CBD is Δ9-CBD.

In some embodiments, the cannabinoid (e.g., CBD) is added to the aerosol precursor composition in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the aerosol precursor composition is no greater than about 1% by weight of the aerosol precursor composition, such as no greater than about 0.5% by weight of the aerosol precursor composition such as no greater than about 0.1% by weight of the aerosol precursor composition such as no greater than about 0.01% by weight of the aerosol precursor composition.

The choice of cannabinoid and the particular percentages thereof which may be present within the aerosol precursor composition will vary depending upon the desired characteristics of the composition.

In some embodiments, the cannabinoid (such as CBD) is present in the aerosol precursor composition in a concentration of at least about 0.001% by weight of the aerosol precursor composition, such as in a range from about 0.001% to about 2% by weight of the aerosol precursor composition. In some embodiments, the cannabinoid (such as CBD) is present in the aerosol precursor composition in a concentration of from about 0.1% to about 1.5% by weight, based on the total weight of the aerosol precursor composition. In some embodiments, the cannabinoid (such as CBD) is present in a concentration from about 0.4% to about 1.5% by weight, based on the total weight of the aerosol precursor composition.

Alternatively, or in addition to a cannabinoid, the other active agent may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

### Terpenes

Other active agents suitable for use in the aerosol precursor composition can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination. Such compounds can be used in the same amounts and ratios noted herein for cannabinoids.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active agent comprises more than one terpene. For example, the active agent may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

Terpenes and/or cannabinoids may be present as an active agent, as an aerosol former, or as a flavorant. The amount of terpene and/or cannabinoid present may vary accordingly based on their intended purpose.

### Flavoring Agent

In some embodiments, the composition as described herein comprises a flavoring agent. As used herein, a “flavoring agent” or “flavorant” is any flavorful or aromatic substance capable of altering the sensory characteristics associated with the liquid aerosol precursor composition or the vapor produced therefrom. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

Flavoring agents may be imitation, synthetic or natural ingredients or blends thereof. Flavoring agents may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents.

Flavorants may further include flavor enhancers, sensorial receptor site activators or stimulators, and trigeminal sensates, As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol.

In some embodiments, the composition comprises a sensate which provides to the user of such composition a cooling effect. Suitable cooling agents include, but are not limited to, menthane, menthone, menthone ketals, menthone glycerol ketals, substituted p-menthanes, acyclic carboxamides, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N-ethyl-p-menthane-3-carboxamide (WS-3), ethyl ester of N-[[5-methyl-2-(1-methylethyl)cyclohexyl]carbonyl]glycine (WS-5), WS-14, N,2,3-trimethyl-2-isopropyl butanamide (WS-23), WS-27, WS-30, (−)-Menthyloxyethanol (Coolact® 5), WS-NA (FEMA4693), WS-116 (FEMA4603), N-ethyl-2,2-diisopropylbutanamide, isopulegol, menthyloxy propane diol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-methylacetic acid N-ethylamide, 1-menthyl-4-hydroxypentanoate, 1-menthyl-3-hydroxybutyrate, N,2,3-trimethyl-2-(1-methylethyl)-butanamide, N-ethyl-trans-2-cis-6-nonadienamide, N,N-dimethyl menthyl succinamide, N-(2-hydroxyethyl)-2,3-dimethyl-2-isopropylbutanamide, substituted p-menthanes, substituted p-menthane-carboxamides, 2-isopropanyl-5-methylcyclohexanol, menthyl ethylene glycol carbonate, menthone glycerol ketals (e.g., menthone 1,2-glycerol ketal), menthone (S)-lactic acid ketal, menthyl acetoacetate, 3-1-menthoxypropane-1,2-diol, menthyl lactate, eucalyptus extract, menthol propylene glycol carbonate, menthol ethylene glycol carbonate, menthol glyceryl ether, N-tert-butyl-p-menthane-3-carboxamide, p-menthane-3-carboxylic acid glycerol ester, methyl-2-isopropyl-bicyclo[2.2.1]heptane-2-carboxamide, (1R,2S,5R)—N-(4-(carbamoylmethyl)phenyl)-menthylcarboxamide, 2-[2-(p-menthan-3-yloxy)ethoxy]ethanol, (1R,2R,4R)-1-(2-Hydroxy-4-methylcyclohexyl)ethenone, 2-(p-tolyloxy)-N-(1H-pyrazol-5-yl)-N-((thiophen-2-yl)methyl)acetamide, menthol methyl ether, menthyl pyrrolidone carboxylate, 2,5-dimethyl-4-(1-pyrrolidinyl)-3(2H)-furanone, cyclic a-keto enamines, and cyclotene derivatives (e.g., 3-methyl-2-(1-pyrrolidinyl)-2-cyclopenten-1-one and 5-methyl-2-(1-pyrrolidinyl)-2-cyclopenten-1-one). Other compounds include the alpha-keto enamines disclosed in U.S. Pat. No. 6,592,884 to Hofmann et al., which is incorporated in its entirety herein. These and other suitable cooling agents are further described in the following U.S. patents, all of which are incorporated in their entirety by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in US Patent Application Publications Nos. 2005/0222256 and 2005/0265930, each of which are incorporated in their entirety by reference hereto. In some embodiments, the cooling agent comprises menthol, eucalyptus, mint, eucolyptol, WS-3, WS-23, WS-5, (1R,2S,5R)—N-(4-(cyanomethyl)phenyl)menthylcarboxamide (Evercool™ 180), (1R,2S,5R)—N-(2-(pyridin-2-yl)ethyl)menthylcarboxamide (Evercool™ 190), or a combination thereof.

In some embodiments, the composition does not comprise a flavoring agent, and comprises only a cooling agent to provide the desired user experience. In some embodiments, the cooling agent is WS-3.

In some embodiments, the composition comprises a modulator or sensate which provides to the user of such composition a warming effect. Suitable warming agents include, but are not limited to, ethers of vanillyl alcohol (e.g., methyl, ethyl, n-propyl, isopropyl, n-butyl, isobutyl, isoamyl, n-hexyl), gingerol, shogaol, paradol, zingerone, capsaicin, dihydrocapsaicin, nordihydrocapsaicin, homocapsaicin, homodihydrocapsaicin, benzyl alcohol, and combinations thereof. In some embodiments, the warming agent comprises vanillyl butyl ether, vanillyl ethyl ether, capsaicin, or a combination thereof.

Flavoring agents may be in any suitable form, for example, a liquid such as an oil, or a solid such as a powder or wax. In some instances, the flavoring agent may be provided in a spray-dried form or a liquid form. In some embodiments, liquid flavoring agents include a carrier/solvent as the predominant component.

The amount of flavoring agent (excluding carrier content such as propylene glycol) utilized in the composition can vary, but is typically up to about 10% by weight, and certain embodiments are characterized by a flavoring agent content of at least about 0.1% by weight, such as about 0.5 to about 10%, about 1 to about 5%, or about 2 to about 4% weight, based on the total weight of the composition.

### Buffering Agents

In some embodiments, the liquid aerosol precursor composition of the present disclosure can comprise buffering agents. Examples of buffering agents that can be used include, but are not limited to, alkali metal buffers such as metal carbonates (e.g., potassium carbonate or sodium carbonate), metal bicarbonates such as sodium bicarbonate, alkali metal citrates, and the like. Non-limiting examples of suitable buffers include alkali or alkaline earth metal (e.g., sodium, potassium, calcium, magnesium) acetates, glycinates, phosphates, glycerophosphates, citrates, carbonates, hydrogen carbonates, borates, and mixtures thereof. In some embodiments, the buffer comprises acetate, phosphate or carbonate, such as an alkali metal acetate, phosphate, or carbonate. In some embodiments, the buffer comprises citrate. In some embodiments, the buffer comprises or is trisodium citrate. In some embodiments, the buffer is a mixture of trisodium citrate and citric acid. In some embodiments, the buffer is a solution of trisodium citrate and citric acid having a pH of about 6.3, such as a solution 0.5 molar in citrate.

When present, the buffering agent is typically present in an amount less than about 5 percent based on the weight of the composition, for example, from about 0.5% to about 5%, such as, e.g., from about 0.75% to about 4%, from about 0.75% to about 3%, or from about 1% to about 2% by weight, based on the total weight of the composition. As described herein above, such buffering agents may be added in a quantity sufficient to provide a specific pH value or range, or to maintain the pH of the composition within a desired range or at a desired value.

### Functional Materials

The aerosol precursor composition may further include one or more functional materials. Functional materials may include one or more of pH regulators, coloring agents, preservatives, stabilizers, and/or antioxidants.

### Form and Properties of Aerosol Precursor Composition

The aerosol precursor composition as described herein is capable of generating an aerosol, for example when heated, irradiated, or energized in any other way. The form of the aerosol precursor composition may vary. The aerosol precursor composition may be, for example, in the form of a liquid, gel, semi-solid, or solid. In some embodiments, the aerosol precursor composition is in the form of a liquid and may be described as a liquid aerosol precursor composition.

The pH of the aerosol precursor composition may vary. In some embodiments, the composition has a pH in a range from about 4.0 to about 9.0, such as from about 4.5 to about 7.0, or from about 5.5 to about 7.0, from about 4.0 to about 5.5, or from about 7.0 to about 9.0. Reference herein to “a composition pH” means the pH of an aqueous solution of the composition prepared by dissolving or suspending 5 grams of composition in 95 grams of water and measuring the pH of the resulting solution with a calibrated pH meter.

In some embodiments, the composition pH is from about 4.5 to about 6.5, for example, from about 4.5, about 5.0, or about 5.5, to about 6.0, or about 6.5. In some embodiments, the desired composition pH is from about 4.5 to about 6.5, and an acid (e.g., an organic acid is described herein) is provided in a quantity sufficient to provide such a pH. In some embodiments, a mineral acid (e.g., hydrochloric acid, sulfuric acid, phosphoric acid, or the like), alone or in combination with an organic acid, is added to adjust the pH of the composition to the desired value. In some embodiments, a buffer (e.g., a buffer as described herein below) is added to the composition to the desired value, and/or to maintain the pH of the composition at the desired value. In some embodiments, the flavoring agent comprises acidic components, and such acidic components may be present in sufficient concentration to achieve a desired pH value of the composition in the absence of further acid addition.

### Aerosol Delivery Devices

Example implementations of the present disclosure are further directed to delivery systems designed to deliver at least one substance to a user, such as to satisfy a particular “consumer moment.” Delivery systems for the aerosol precursor composition provided herein may take many forms. Examples of suitable delivery systems include aerosol provision systems such as powered aerosol provision systems designed to release one or more substances or compounds from an aerosol generating material (e.g., an aerosol precursor composition as disclosed herein, such as a liquid aerosol precursor composition) without combusting the aerosol generating material. These aerosol provision systems may at times be referred to as non-combustible aerosol provision systems, aerosol delivery devices or the like. The substance may include constituents that impart a physiological effect on the user, a sensorial effect on the user, or both.

An example of a suitable aerosol provision system includes vapor products. Vapor products are commonly known as “electronic cigarettes,” “e-cigarettes” or electronic nicotine delivery systems (ENDS), although the aerosol-generating material need not include nicotine. Many vapor products are designed to heat a liquid material to generate an aerosol. Other vapor products are designed to break up an aerosol generating material into an aerosol without heating, or with only secondary heating.

Accordingly, in another aspect is provided an aerosol delivery device. The aerosol delivery device according to the present disclosure may take on a variety of embodiments, as discussed in detail below. However, typically, the use of the aerosol delivery device by a consumer will be similar in scope. The foregoing description of the aerosol precursor composition is applicable to the various embodiments described through minor modifications, which are apparent to the person of skill in the art in light of the further disclosure provided herein. The description of use, however, is not intended to limit the use of the aerosol precursor composition as disclosed herein but is provided to comply with all necessary requirements of disclosure herein.

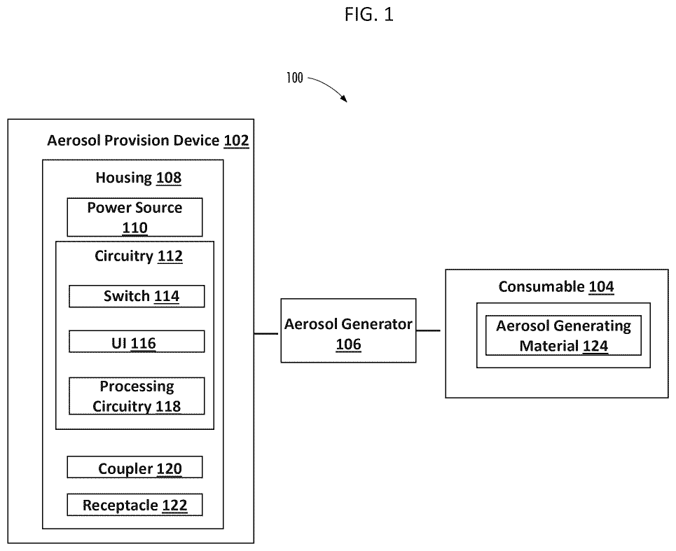

FIG. 1 is a block diagram of an aerosol delivery device 100 according to some example implementations, incorporating an aerosol generating material 124, comprising an aerosol precursor composition as disclosed herein, such as a liquid aerosol precursor composition. In various examples, the aerosol delivery device 100 may be a vapor product. The aerosol delivery device 100 includes one or more of each of a number of components including, for example, an aerosol provision device 102, and a consumable 104 (sometimes referred to as an article) for use with the aerosol provision device 102. The aerosol generating material 124 can be included within an article of a consumable 104, part or all of which is intended to be consumed during use by a user. The aerosol delivery device 100 may include one or more consumables 104, and each consumable 104 may include one or more aerosol generating material 124 (which can comprise or can be a liquid aerosol precursor composition as described herein, or which can be alternative types of compositions).

The aerosol delivery device 100 also includes an aerosol generator 106. The aerosol generator 106 (also referred to as an atomizer, aerosolizer, or aerosol production component) is configured to energize the aerosol generating material 124 to generate an aerosol, or otherwise cause generation of an aerosol from the aerosol generating material 124. In various implementations, the aerosol generator 106 may be part of the aerosol provision device 102 or the consumable 104. In other implementations, the aerosol generator 106 may be separate from the aerosol provision device 102 and the consumable 104, and removably engaged with the aerosol provision device 102 and/or the consumable 104.

In various examples, the aerosol provision system 100 and its components including the aerosol provision device 102 and the consumable 104 may be reusable or single use. In some examples, the aerosol delivery device 100 including both the aerosol provision device 102 and the consumable 104 may be single use. In some examples, the aerosol provision device 102 may be reusable, and the consumable 104 may be reusable (e.g., refillable) or single use (e.g., replaceable). In yet further examples, the consumable 104 may be both refillable and replaceable. In examples in which the aerosol generator 106 is part of the aerosol provision device 102 or the consumable 104, the aerosol generator 106 may be reusable or single use in the same manner as the aerosol provision device 102 or the consumable 104.

The consumable may include an aerosol generating material transfer component (also referred to as a liquid transport element) configured to transport aerosol generating material to the aerosol generator 106. The aerosol generating material transfer component may be adapted to wick or otherwise transport aerosol generating material via capillary action. In some examples, the aerosol generating material transfer component may include a microfluidic chip, a micro pump or other suitable component to transport aerosol generating material (e.g., a liquid aerosol precursor composition as described herein).

In some example implementations, the aerosol provision device 102 may include a housing 108 with a power source 110 and circuitry 112. The power source is configured to provide a source of power to the aerosol provision device and thereby the aerosol provision system 100. The power source may be or include, for example, an electric power source such as a non-rechargeable battery or a rechargeable battery, solid-state battery (SSB), lithium-ion battery, supercapacitor, or the like.

The circuitry 112 may be configured to enable one or more functionalities (at times referred to as services) of the aerosol provision device 102 and thereby the aerosol provision system 100. The circuitry includes electronic components, and in some examples one or more of the electronic components may be formed as a circuit board such as a printed circuit board (PCB).

In some examples, the circuitry 112 includes at least one switch 114 that may be directly or indirectly manipulated by a user to activate the aerosol provision device 102 and thereby the aerosol delivery device 100. The switch may be or include a pushbutton, touch-sensitive surface or the like that may be operated manually by a user. Additionally, or alternatively, the switch may be or include a sensor configured to sense one or more process variables that indicate use of the aerosol provision device or aerosol provision system. One example is a flow sensor, pressure sensor, pressure switch or the like that is configured to detect airflow or a change in pressure caused by airflow when a user draws on the consumable 104.

The switch 114 may provide user interface functionality. In some examples, the circuitry 112 may include a user interface (UI) 116 that is separate from or that is or includes the switch. The UI may include one or more input devices and/or output devices to enable interaction between the user and the aerosol provision device 102. As described above with respect to the switch, examples of suitable input devices include pushbuttons, touch-sensitive surfaces and the like. The one or more output devices generally include devices configured to provide information in a human-perceptible form that may be visual, audible or tactile/haptic. Examples of suitable output devices include light sources such as light-emitting diodes (LEDs), quantum dot-based LEDs and the like. Other examples of suitable output devices include display devices (e.g., electronic visual displays), touchscreens (integrated touch-sensitive surface and display device), loudspeakers, vibration motors and the like.

In some examples, the circuitry 112 includes processing circuitry 118 configured to perform data processing, application execution, or other processing, control or management services according to one or more example implementations. The processing circuitry may include a processor embodied in a variety of forms such as at least one processor core, microprocessor, coprocessor, controller, microcontroller or various other computing or processing devices including one or more integrated circuits such as, for example, an ASIC (application specific integrated circuit), an FPGA (field programmable gate array), some combination thereof, or the like. In some examples, the processing circuitry may include memory coupled to or integrated with the processor, and which may store data, computer program instructions executable by the processor, some combination thereof, or the like.

As also shown, in some examples, the housing 108 and thereby the aerosol provision device 102 may also include a coupler 120 and/or a receptacle 122 structured to engage and hold the consumable 104, and thereby couple the aerosol provision device 102 with the consumable 104. The coupler may be or include a connector, fastener or the like that is configured to connect with a corresponding coupler of the consumable, such as by a press fit (or interference fit) connection, threaded connection, magnetic connection or the like. The receptacle may be or include a reservoir, tank, container, cavity, receiving chamber or the like that is structured to receive and contain the consumable or at least a portion of the consumable.

In some examples, the aerosol generator 106 may be powered by the power source 110 under control of the circuitry 112 to energize the aerosol-generating material to generate an aerosol. In some example implementations, the aerosol generator 106 is an electric heater configured to perform electric heating in which electrical energy from the power source 110 is converted to heat energy, which the aerosol generating material 124 is subject to so as to release one or more volatiles from the aerosol generating material 124 to form an aerosol. Examples of suitable forms of electric heating include resistance (Joule) heating, induction heating, dielectric and microwave heating, radiant heating, arc heating and the like. More particular examples of suitable electric heaters include resistive heating elements such as wire coils, flat plates, prongs, micro heaters or the like.

In some example implementations, the aerosol generator 106 is configured to cause an aerosol to be generated from the aerosol generating material 124 without heating, or with only secondary heating. For example, the aerosol generator 106 may be configured to subject the aerosol generating material 124 to one or more of increased pressure, vibration, or electrostatic energy. More particular examples of these aerosol generators include jet nebulizers, ultrasonic wave nebulizers, vibrating mesh technology (VMT) nebulizers, surface acoustic wave (SAW) nebulizers, and the like.

A jet nebulizer is configured to use compressed gas (e.g., air, oxygen) to break up aerosol generating material into an aerosol, and an ultrasonic wave nebulizer is configured to use ultrasonic waves to break up aerosol generating material into an aerosol. A VMT nebulizer includes a mesh, and a piezo material (e.g., piezoelectric material, piezomagnetic material) that may be driven to vibrate and cause the mesh to break up aerosol generating material into an aerosol. A SAW nebulizer is configured to use surface acoustic waves or Rayleigh waves to break up aerosol generating material into an aerosol.

In some examples, the aerosol generator 106 may include a susceptor. The susceptor is a material that is heatable by penetration with a varying magnetic field generated by a magnetic field generator that may be separate from or part of the aerosol generator. The susceptor may be an electrically conductive material, so that penetration thereof with a varying magnetic field causes induction heating of the heating material. The heating material may be magnetic material, so that penetration thereof with a varying magnetic field causes magnetic hysteresis heating of the heating material. The susceptor in some examples may be both electrically conductive and magnetic, so that the susceptor of these examples is heatable by both heating mechanisms.

The aerosol delivery device 100 and its components including the aerosol provision device 102, consumable 104, and aerosol generator 106 may be manufactured with any of a number of different form factors, and with additional or alternative components relative to those described above.

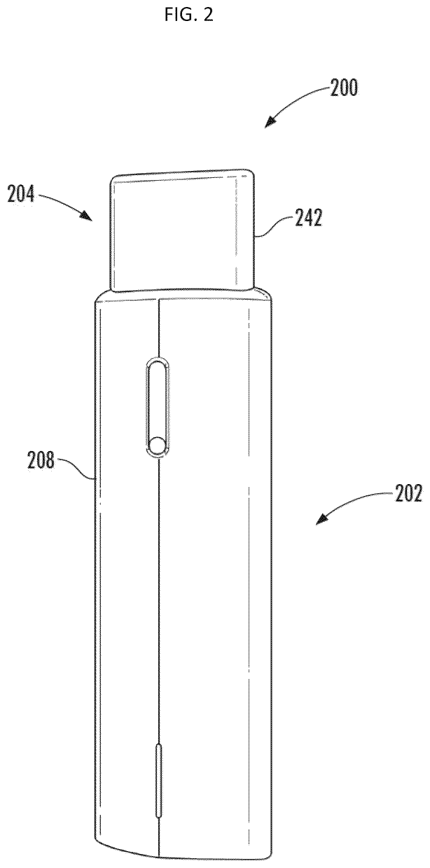

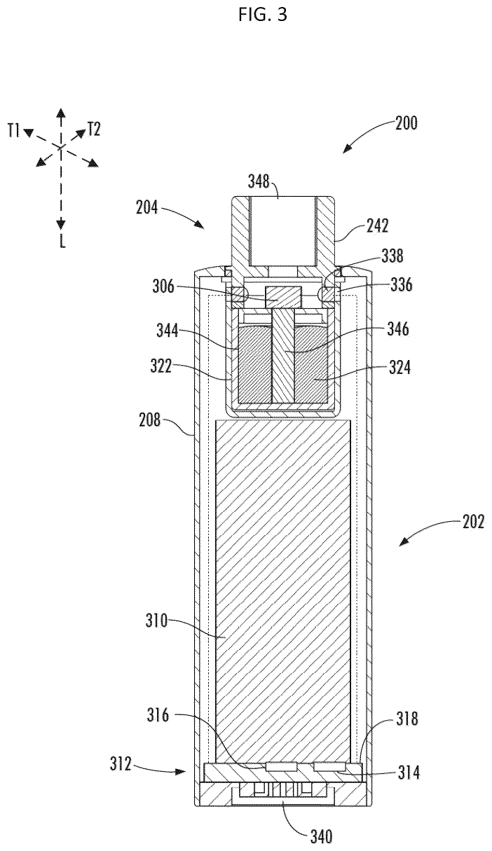

FIGS. 2 and 3 illustrate an aerosol delivery device 200 in the form of a vapor product, and that in some example implementations may correspond to the aerosol delivery device 100. As shown, the aerosol delivery device 200 may include an aerosol provision device 202 (also referred to as a control body or power unit) and a consumable 204 (also referred to as a cartridge or tank), which may correspond to respectively the aerosol provision device 102 and the consumable 104. The aerosol delivery device 200 and in particular the consumable 204 may also include an aerosol generator corresponding to the aerosol generator 106, and in the form of an electric heater 306 such as a heating element like a metal wire coil configured to convert electrical energy to heat energy through resistance (Joule) heating. The aerosol provision device 202 and the consumable 204 can be permanently or detachably aligned in a functioning relationship. FIGS. 2 and 3 illustrate respectively a perspective view and a partially cut-away side view of the aerosol provision system in a coupled configuration.

As seen in FIG. 2 and the cut-away view illustrated in FIG. 3, the aerosol provision device 202 and consumable 204 each include a number of respective components. The components illustrated in FIG. 3 are representative of the components that may be present in an aerosol provision device and consumable and are not intended to limit the scope of components that are encompassed by the present disclosure.

The aerosol provision device 202 may include a housing 208 (sometimes referred to as an aerosol provision device shell) that may include a power source 310. The housing may also include circuitry 312 with a switch in the form of a sensor 314, a user interface including a light source 316 that may be illuminated with use of the aerosol provision system 200, and processing circuitry 318 (also referred to as a control component). The housing may also include a receptacle in the form of a consumable receiving chamber 322 structured to engage and hold the consumable 204. The consumable 204 may include an aerosol generating material 324 that may correspond to aerosol generating material 124 as described herein.

As also seen in FIG. 3, the aerosol provision device 202 may also include electrical connectors 336 positioned in the consumable receiving chamber 322 configured to electrically couple the circuitry and thereby the aerosol provision device 202 with the consumable 204, and in particular electrical contacts 338 on the consumable 204. In this regard, the electrical connectors and electrical contacts may form a connection interface of the aerosol provision device and consumable. As also shown, the aerosol provision device 202 may include an external electrical connector 340 to connect the aerosol provision device with one or more external devices. Examples of suitable external electrical connectors include USB connectors, proprietary connectors such as Apple's Lightning connector, and the like.

In various examples, the consumable 204 includes a tank portion and a mouthpiece portion. The tank portion and the mouthpiece portion may be integrated or permanently fixed together, or the tank portion may itself define the mouthpiece portion (or vice versa). In other examples, the tank portion and the mouthpiece portion may be separate and removably engaged with one another.

The tank portion and/or mouthpiece portion of consumable 204 may be separately defined in relation to a longitudinal axis (L), a first transverse axis (T1) that is perpendicular to the longitudinal axis, and a second transverse axis (T2) that is perpendicular to the longitudinal axis and is perpendicular to the first transverse axis. The consumable 204 can be formed of a housing 242 (sometimes referred to as the consumable shell) enclosing a reservoir 344 (in the tank portion) configured to retain the aerosol generating material 324. In some examples, the consumable may include an aerosol generator, such as electric heater 306 in the illustrated example. In some examples, the electrical connectors 336 on the aerosol provision device 202 and electrical contacts 338 on the consumable may electrically connect the electric heater with the power source 310 and/or circuitry 312 of the aerosol provision device.

As shown, in some examples, the reservoir 344 may be in fluid communication with an aerosol generating material transfer component 346 adapted to wick or otherwise transport aerosol generating material 324 stored in the reservoir housing to the electric heater 306. At least a portion of the aerosol generating material transfer component may be positioned proximate (e.g., directly adjacent, adjacent, in close proximity to, or in relatively close proximity to) the electric heater. The aerosol generating material transfer component may extend between the electric heater and the aerosol generating material stored in the reservoir, and at least a portion of the electric heater may be located above a proximal end the reservoir. For the purposes of the present disclosure, it should be understood that the term “above” in this particular context should be interpreted as meaning toward a proximal end of the reservoir and/or the consumable 204 in direction substantially along the longitudinal axis (L). Other arrangements of the aerosol generating material transfer component are also contemplated within the scope of the disclosure. For example, in some example implementations, the aerosol generating material transfer component may be positioned proximate a distal end of the reservoir and/or arranged transverse to the longitudinal axis (L).

The electric heater 306 and aerosol generating material transfer component 346 may be configured as separate elements that are fluidly connected, the electric heater and aerosol-generating material transfer component or may be configured as a combined element. For example, in some implementations an electric heater may be integrated into an aerosol generating material transfer component. Moreover, the electric heater and the aerosol-generating material transfer component may be formed of any construction as otherwise described herein. In some examples, a valve may be positioned between the reservoir 344 and electric heater and configured to control an amount of aerosol generating material 324 passed or delivered from the reservoir to the electric heater.

An opening 348 may be present in the housing 242 (e.g., at the mouth end of the mouthpiece portion) to allow for egress of formed aerosol from the consumable 204.

As indicated above, the circuitry 312 of the aerosol provision device 202 may include a number of electronic components, and in some examples may be formed of a circuit board such as a PCB that supports and electrically connects the electronic components. The sensor 314 (switch) may be one of these electronic components positioned on the circuit board. In some examples, the sensor may comprise its own circuit board or other base element to which it can be attached. In some examples, a flexible circuit board may be utilized. A flexible circuit board may be configured into a variety of shapes. In some examples, a flexible circuit board may be combined with, layered onto, or form part or all of a heater substrate.

In some examples, the reservoir 344 may be a container for storing the aerosol generating material 324. In some examples, the reservoir may be or include a fibrous reservoir. For example, the reservoir can comprise one or more layers of nonwoven fibers substantially formed into the shape of a tube encircling the interior of the housing 242, in this example. The aerosol generating material 324 may be retained in the reservoir. Liquid components, for example, may be sorptively retained by the reservoir. The reservoir may be in fluid connection with the aerosol-generating material transfer component 346. The aerosol generating material transfer component may transport the aerosol generating material 324 stored in the reservoir via capillary action—or via a micro pump—to the electric heater 306. As such, the electric heater is in a heating arrangement with the aerosol-generating material transfer component.

In use, when a user draws on the aerosol provision system 200, airflow is detected by the sensor 314, and the electric heater 306 is activated to energize the aerosol generating material 324 to generate an aerosol. Drawing upon the mouth end of the aerosol provision system causes ambient air to enter and pass through the aerosol provision system. In the consumable 204, the drawn air combines with the aerosol that is whisked, aspirated or otherwise drawn away from the electric heater and out the opening 348 in the mouth end of the aerosol provision system.

Again, as shown in FIGS. 2 and 3, the aerosol generator of the aerosol provision system 200 is an electric heater 306 designed to heat the aerosol generating material 324 to generate an aerosol.

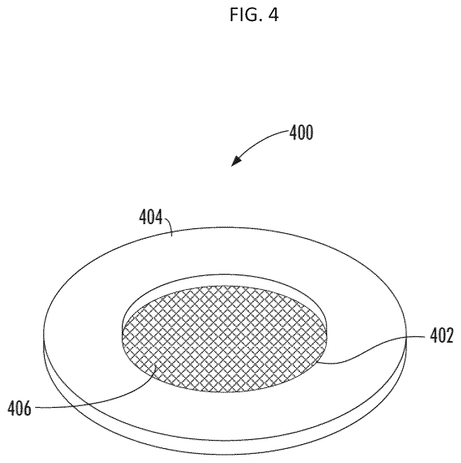

In other implementations, the aerosol generator is designed to break up the aerosol generating material without heating, or with only secondary heating. FIG. 4 illustrates a nebulizer 400 that may be used to implement the aerosol generator of an aerosol provision system, according to some these other example implementations. As shown in FIG. 4, the nebulizer 400 includes a mesh plate 402 and a piezo material 404 that may be affixed to one another. The piezo material may be driven to vibrate and cause the mesh plate to break up aerosol-generating material into an aerosol. In some examples, the nebulizer may also include a supporting component located on a side of the mesh plate opposite the piezo material to increase the longevity of the mesh plate, and/or an auxiliary component between the mesh plate and the piezo material to facilitate interfacial contact between the mesh plate and the piezo material.

In various example implementations, the mesh plate 402 may have a variety of different configurations. The mesh plate may have a flat profile, a domed shape (concave or convex with respect to the aerosol-generating material), or a flat portion and a domed portion. The mesh plate defines a plurality of perforations 406 that may be substantially uniform or vary in size across a perforated portion of the mesh plate. The perforations may be circular openings or non-circular openings (e.g., oval, rectangular, triangular, regular polygon, irregular polygon). In three-dimensions, the perforations may have a fixed cross section such as in the case of cylindrical perforations with a fixed circular cross section, or a variable cross section such as in the case of truncated cone perforations with a variable circular cross section. In other implementations, the perforations may be tetragonal or pyramidal.

The piezo material 404 may be or include a piezoelectric material or a piezomagnetic material. A piezoelectric material may be coupled to circuitry configured to produce an oscillating electric signal to drive the piezoelectric material to vibrate. For a piezomagnetic material, the circuitry may produce a pair of antiphase, oscillating electric signals to drive a pair of magnets to produce antiphase, oscillating magnetic fields that drives the piezomagnetic material to vibrate.

The piezo material 404 may be affixed to the mesh plate 402, and vibration of the piezo material may in turn cause the mesh plate to vibrate. The mesh plate may be in contact with or immersed in aerosol-generating material, in sufficient proximity of aerosol-generating material, or may otherwise receive aerosol-generating material via an aerosol-generating material transfer component. The vibration of the mesh plate, then, may cause the aerosol-generating material to pass through the perforations 406 that break up the aerosol-generating material into an aerosol. More particularly, in some examples, aerosol-generating material may be driven through the perforations 406 in the vibrating mesh plate 402 resulting in aerosol particles. In other examples in which the mesh plate is in contact with or immersed in aerosol-generating material, the vibrating mesh plate may create ultrasonic waves within aerosol-generating material that cause formation of an aerosol at the surface of the aerosol-generating material.

In addition, the disclosure provides kits that provide a variety of components as described herein. For example, a kit may comprise a control body with one or more aerosol generating components/cartridges (including at least one such component comprising a liquid aerosol precursor composition as provide herein). In further embodiments, a kit may comprise a plurality of aerosol generating components/cartridges. In the above embodiments, the aerosol generating components or the control bodies may be provided with a heating member inclusive thereto. A kit may further comprise one or more charging components and/or one or more batteries. The kits may further include a case (or other packaging, transporting, or storage component) that accommodates one or more of the further kit components. The case could be a reusable hard or soft container. Further, the case could be simply a box or other packaging structure.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

### EXAMPLES

### Example 1. Preparation of a Liquid Aerosol Precursor Composition Comprising 2-methyl-5-(1-methylpyrrolidin-2-yl)pyridine

A liquid aerosol precursor composition according to an embodiment of the disclosure is prepared having the formulation is provided in Table 2. The composition is prepared by combining the components of Table 2 and mixing thoroughly to form a homogenous solution.

TABLE 2
Formulation of a liquid aerosol composition

| Component | Percent by Weight |
|---|---|
| 2-methyl-5-(1-methylpyrrolidin- | 0.1-1.0 (approximately 1% to 10% by |
| 2-yl)pyridine | weight of a 12% solution of 2-methyl- |
| | 5-(1-methylpyrrolidin-2-yl)pyridine) |
| propylene glycol | 20-40 |
| glycerin | 24-48 |
| organic acid (e.g., lactic acid) | 0-0.5 (0 to 1 molar equivalents) |
| flavor | 20-40 |

### Examples 2A-2X. Preparation of Flavored Liquid Aerosol Precursor Compositions Comprising 2-methyl-5-(1-methylpyrrolidin-2-yl)pyridine

A series of liquid aerosol precursor compositions according to embodiments of the disclosure were prepared having the formulations provided in Table 3. The compositions were prepared by combining a glycerin solution containing 12% by weight of 2-methyl-5-(1-methylpyrrolidin-2-yl)pyridine, propylene glycol (PG), glycerin (VG), and flavoring agents (identified as flavors 1 to 15) in the quantities provided in Table 3 and mixing thoroughly to form a homogenous solution. The weight percent of each respective flavoring agent is shown in the table. The total % by weight of VG and PG listed in the table is exclusive of any VG or PG in the flavoring agent or 2-methyl-5-(1-methylpyrrolidin-2-yl)pyridine solution. However, the PG to VG weight ratio is based on the approximated total amount of VG and PG in the liquid aerosol composition including the flavoring agent and the glycerin content of the 2-methyl-5-(1-methylpyrrolidin-2-yl)pyridine solution.

TABLE 3
Formulations of liquid aerosol compositions

| | | % by weight of 2- | Molar | Total % | |
|---|---|---|---|---|---|
| | | methyl-5-(1- | Equivalents | by Weight | PG to VG |
| Example | | methylpyrrolidin- | of Lactic | of PG | Weight |
| # | Flavor # | 2-yl)pyridine | Acid | and VG | Ratio |
| 2A | 1 (30% by weight) | 0.5 | 1 | 65.5 | 1.5:1 |
| 2B | 2 (30% by weight) | 0.5 | 1 | 65.5 | 1.5:1 |
| 2C | 3 (30% by weight) | 0.5 | 1 | 65.5 | 1.5:1 |
| 2D | 4 (30% by weight) | 0.75 | 1 | 63.4 | 1.5:1 |
| 2E | 5 (30% by weight) | 0.75 | 1 | 63.4 | 1.5:1 |
| 2F | 6 (20% by weight) | 0.5 | 1 | 75.5 | 1.5:1 |
| 2G | 7 (30% by weight) | 0.5 | 1 | 65.5 | 1.5:1 |
| 2H | 8 (30% by weight) | 0.5 | 1 | 65.5 | 1.5:1 |
| 2I | 9 (30% by weight) | 0.5 | 1 | 65.5 | 1:1 |
| 2J | 9 (30% by weight) | 0.5 | 1 | 65.5 | 1.5:1 |
| 2K | 10 (30% by weight) | 0.5 | 1 | 65.5 | 1.5:1 |
| 2L | 10 (30% by weight) | 0.75 | 1 | 65.5 | 1.5:1 |
| 2M | 11 (30% by weight) | 0.5 | 1 | 63.4 | 1.5:1 |
| 2N | 12 (5% by weight) | 0.15 | 0 | 93.8 | 1:1 |
| 2O | 12 (5% by weight) | 0.15 | 0.5 | 93.8 | 1:1 |
| 2P | 12 (5% by weight) | 0.15 | 1 | 93.6 | 1:1 |
| 2Q | 12 (5% by weight) | 0.25 | 0.5 | 92.8 | 1:1 |
| 2R | 12 (5% by weight) | 0.25 | 1 | 92.7 | 1:1 |
| 2S | 12 (5% by weight) | 0.5 | 1 | 90.5 | 1:1 |
| 2T | 12 (5% by weight) | 0.75 | 1 | 88.4 | 1:1 |
| 2U | 13 (30% by weight) | 0.5 | 1 | 65.5 | 1.5:1 |
| 2V | 14 (30% by weight) | 0.5 | 1 | 65.5 | 1.5:1 |
| 2W | 15 (30% by weight) | 0.5 | 1 | 65.5 | 1:1 |
| 2X | 15 (30% by weight) | 0.5 | 1 | 65.5 | 1.5:1 |

### Example 3. Preparation of a Reference Liquid Aerosol Precursor Composition Comprising Nicotine

A reference liquid aerosol precursor composition was prepared having the formulation provided in Table 4. The composition was prepared by combining the components of Table 4 and mixing thoroughly to form a homogenous solution.

TABLE 4
Formulation of a reference liquid aerosol composition

| Component | Percent by Weight |
|---|---|
| Nicotine (10% solution in | 50% (5% nicotine by |
| glycerin) | weight) |
| Propylene glycol | 41.9 |
| organic acid (e.g., lactic acid) | 1 molar equivalent |
| | (3.15% by weight) |
| Flavor (#12) | 5 |

### Example 4. Sensory Evaluation of Compositions

The compositions of Examples 2 and 3 were evaluated for the sensory qualities of the vapor formed therefrom by a sensory evaluation panel. The vaporization was performed using three different devices (denoted as 1, 2, and 3). The evaluations were performed as a “roundtable” assessment in which 10-11 participants took no more than 10 puffs on 4 different variants according to Table 3, with the option to compare to a 5% nicotine control product (Example 3). The session(s) lasted approximately 1 hour. Factors such as taste, throat impact, aftertaste, and harshness were evaluated on a scale of 1-7 (low to high degree). Additional categories were rated on a scale of 1-5, such as overall taste and aftertaste, satisfaction, and preference relative to the e-liquid normally used by the participant. The results are expressed in Table 5 as the average score in each category, along with the standard deviation (SD) and relative standard deviation (RSD) for each average.

Overall, the data demonstrate that the compositions of the disclosure comprising 2-methyl-5-(1-methylpyrrolidin-2-yl)pyridine were able to match the throat impact and other desired sensations of the control nicotine composition, but at much lower inclusion levels of active (0.15 to 0.75% by weight 2-methyl-5-(1-methylpyrrolidin-2-yl)pyridine versus 5% by weight nicotine). Further, there was an overall smoothness noted relative to nicotine without inclusion of excess or equimolar organic acid quantities. These effects were observed across multiple flavors and with three different devices.

TABLE 5
Sensory Data

| Example | | | | Ideal | | Ideal | Throat | Ideal Throat | |
|---|---|---|---|---|---|---|---|---|---|
| # | Device # | Statistics | Smoothness | Smoothness | Harshness | Harshness | Impact | Impact | Aftertaste |
| 3 | 1 | AVG | 4.9 | 5.3 | 3.5 | 2.6 | 4.4 | 3.6 | 2.6 |
| (Control) | | SD | 1.2 | 1.2 | 1.6 | 1.4 | 0.9 | 1.3 | 0.9 |
| 2N | | RSD | 25.6 | 22.2 | 45.8 | 53.6 | 20.9 | 35.9 | 34.9 |
| | 1 | AVG | 6.1 | 5.0 | 1.9 | 2.8 | 1.4 | 3.3 | 2.4 |
| | | SD | 1.0 | 1.1 | 1.1 | 1.3 | 0.5 | 0.7 | 1.3 |
| | | RSD | 16.2 | 21.4 | 60.1 | 46.6 | 37.6 | 21.8 | 54.8 |
| 2P | 1 | AVG | 6.5 | 5.1 | 1.2 | 2.8 | 1.6 | 3.6 | 3.0 |
| | | SD | 0.6 | 1.3 | 0.4 | 1.6 | 1.0 | 1.2 | 1.9 |
| | | RSD | 9.4 | 24.8 | 36.1 | 56.3 | 65.2 | 34.8 | 62.4 |
| 2Q | 1 | AVG | 5.8 | 5.3 | 2.6 | 2.9 | 2.6 | 3.3 | 2.5 |
| | | SD | 0.9 | 1.2 | 1.1 | 1.1 | 1.0 | 1.2 | 0.9 |
| | | RSD | 15.4 | 22.2 | 40.4 | 39.2 | 36.4 | 35.8 | 37.0 |
| 2R | 1 | AVG | 5.9 | 5.3 | 2.1 | 2.8 | 2.1 | 3.4 | 2.9 |
| | | SD | 1.4 | 1.3 | 1.1 | 1.3 | 0.8 | 0.7 | 0.6 |
| | | RSD | 23.1 | 24.4 | 52.5 | 46.6 | 39.3 | 22.0 | 22.3 |
| 2S | 1 | AVG | 5.2 | 5.1 | 3.3 | 3.0 | 3.4 | 3.4 | 3.0 |
| | | SD | 1.1 | 1.2 | 0.9 | 1.1 | 1.2 | 1.1 | 1.2 |
| | | RSD | 20.6 | 24.3 | 27.3 | 35.6 | 35.2 | 31.4 | 39.8 |
| 2T | 1 | AVG | 5.2 | 5.0 | 3.1 | 2.8 | 3.3 | 3.3 | 2.2 |
| | | SD | 1.2 | 1.1 | 1.1 | 1.2 | 1.0 | 1.2 | 1.0 |
| | | RSD | 22.6 | 21.9 | 36.1 | 41.3 | 30.4 | 36.1 | 45.4 |
| 2W | 1 | AVG | 5.8 | 5.0 | 1.6 | 2.4 | 2.0 | 3.2 | 3.6 |
| | | SD | 0.8 | 1.2 | 0.9 | 1.1 | 0.7 | 0.8 | 0.9 |
| | | RSD | 14.4 | 24.5 | 55.9 | 47.5 | 35.4 | 26.1 | 24.8 |
| 2I | 1 | AVG | 5.0 | 5.2 | 2.0 | 1.8 | 2.8 | 3.2 | 4.0 |
| | | SD | 1.2 | 1.1 | 0.7 | 0.8 | 1.3 | 0.8 | 1.2 |
| | | RSD | 24.5 | 21.1 | 35.4 | 46.5 | 46.6 | 26.1 | 30.6 |
| 2S | 2 | AVG | 3.8 | 4.7 | 4.1 | 3.3 | 3.7 | 3.7 | 3.2 |
| | | SD | 1.3 | 0.8 | 0.7 | 1.2 | 0.8 | 0.5 | 1.2 |
| | | RSD | 33.5 | 17.5 | 16.3 | 36.3 | 22.3 | 14.1 | 36.9 |
| 2S | 1 | AVG | 5.3 | 4.7 | 2.4 | 3.3 | 2.3 | 3.5 | 2.7 |
| | | SD | 1.0 | 0.8 | 1.1 | 1.2 | 1.0 | 0.5 | 0.5 |
| | | RSD | 19.4 | 17.5 | 46.1 | 36.3 | 44.3 | 15.6 | 19.4 |
| 2D | 2 | AVG | 4.8 | 5.0 | 3.0 | 3.0 | 3.5 | 3.4 | 3.4 |
| | | SD | 0.8 | 1.0 | 1.6 | 1.6 | 1.3 | 1.1 | 1.1 |
| | | RSD | 17.4 | 20.0 | 54.4 | 52.7 | 36.9 | 33.5 | 33.5 |
| 2D | 1 | AVG | 5.4 | 5.0 | 2.2 | 2.8 | 2.4 | 3.4 | 2.4 |
| | | SD | 0.9 | 1.0 | 1.1 | 1.3 | 1.1 | 1.1 | 0.9 |
| | | RSD | 16.6 | 20.0 | 49.8 | 46.6 | 47.5 | 33.5 | 37.3 |
| 2E | 3 | AVG | 4.7 | 5.0 | 3.3 | 2.8 | 3.8 | 3.7 | 4.1 |
| | | SD | 0.8 | 0.9 | 1.4 | 1.2 | 1.2 | 1.0 | 1.1 |
| | | RSD | 17.5 | 17.9 | 41.0 | 41.3 | 30.5 | 28.2 | 27.3 |
| 2E | 1 | AVG | 6.0 | 5.2 | 2.6 | 2.8 | 3.0 | 3.6 | 3.2 |
| | | SD | 0.7 | 0.8 | 1.5 | 1.5 | 1.6 | 1.1 | 1.3 |
| | | RSD | 11.8 | 16.1 | 58.3 | 53.0 | 52.7 | 31.7 | 40.7 |
| 2L | 3 | AVG | 5.2 | 5.3 | 3.0 | 2.3 | 3.4 | 2.8 | 4.2 |
| | | SD | 1.5 | 1.2 | 1.3 | 1.2 | 0.9 | 0.8 | 1.5 |
| | | RSD | 28.5 | 22.7 | 42.2 | 51.9 | 26.9 | 26.6 | 35.3 |
| 2L | 1 | AVG | 5.5 | 5.3 | 2.3 | 2.3 | 2.8 | 3.0 | 3.2 |
| | | SD | 1.4 | 1.2 | 1.5 | 1.2 | 1.0 | 0.6 | 1.3 |
| | | RSD | 25.1 | 22.7 | 64.5 | 51.9 | 34.7 | 21.1 | 42.0 |
| 2V | 3 | AVG | 5.2 | 4.2 | 2.0 | 2.8 | 2.8 | 3.3 | 4.0 |
| | | SD | 1.2 | 1.2 | 0.9 | 1.2 | 1.0 | 1.0 | 1.4 |
| | | RSD | 22.6 | 28.1 | 44.7 | 41.3 | 34.7 | 31.0 | 35.4 |
| 2C | 3 | AVG | 5.2 | 4.8 | 2.7 | 2.8 | 3.5 | 3.5 | 3.8 |
| | | SD | 1.0 | 0.8 | 1.6 | 1.2 | 1.6 | 1.0 | 1.7 |
| | | RSD | 19.0 | 15.6 | 61.2 | 41.3 | 46.9 | 30.0 | 44.9 |
| 2M | 3 | AVG | 5.4 | 5.0 | 2.4 | 2.6 | 3.0 | 3.2 | 3.4 |
| | | SD | 1.3 | 0.7 | 2.1 | 1.1 | 1.7 | 0.8 | 1.1 |
| | | RSD | 24.8 | 14.1 | 86.4 | 43.9 | 57.7 | 26.1 | 33.5 |
| 2G | 3 | AVG | 5.8 | 4.8 | 2.2 | 2.6 | 3.2 | 2.8 | 2.6 |
| | | SD | 1.3 | 0.8 | 0.8 | 1.1 | 1.3 | 0.8 | 1.1 |
| | | RSD | 21.9 | 17.4 | 38.0 | 43.9 | 40.7 | 29.9 | 43.9 |
| 2K | 3 | AVG | 5.0 | 4.6 | 2.4 | 2.8 | 3.6 | 3.8 | 3.4 |
| | | SD | 1.0 | 0.9 | 1.1 | 1.3 | 1.5 | 0.8 | 0.5 |
| | | RSD | 20.0 | 19.4 | 47.5 | 46.6 | 42.1 | 22.0 | 16.1 |
| 2J | 3 | AVG | 4.8 | 4.4 | 2.6 | 3.0 | 3.6 | 3.8 | 3.6 |
| | | SD | 1.3 | 1.1 | 1.1 | 1.2 | 1.3 | 0.8 | 0.9 |
| | | RSD | 27.2 | 25.9 | 43.9 | 40.8 | 37.3 | 22.0 | 24.8 |
| 2U | 3 | AVG | 5.3 | 5.3 | 2.1 | 2.7 | 3.1 | 3.6 | 3.9 |
| | | SD | 1.1 | 1.1 | 1.1 | 1.1 | 1.8 | 0.8 | 1.3 |
| | | RSD | 21.1 | 21.1 | 49.9 | 41.0 | 56.4 | 22.0 | 34.9 |
| | 3 | AVG | 5.8 | 5.3 | 2.7 | 2.7 | 3.3 | 3.3 | 3.1 |
| | | SD | 1.3 | 1.1 | 1.5 | 1.1 | 1.6 | 1.0 | 0.7 |
| | | RSD | 23.3 | 21.1 | 55.1 | 41.0 | 48.8 | 28.9 | 22.0 |
| 2A | 3 | AVG | 5.8 | 5.2 | 2.3 | 2.7 | 3.2 | 3.3 | 3.3 |
| | | SD | 1.2 | 1.0 | 1.0 | 1.2 | 1.0 | 1.0 | 1.0 |
| | | RSD | 20.0 | 19.0 | 44.3 | 45.4 | 31.0 | 31.0 | 31.0 |
| 2H | 3 | AVG | 5.8 | 5.2 | 2.3 | 2.8 | 2.8 | 3.3 | 3.2 |
| | | SD | 1.2 | 1.0 | 1.0 | 1.2 | 0.8 | 1.0 | 1.3 |
| | | RSD | 20.0 | 19.0 | 44.3 | 41.3 | 26.6 | 31.0 | 40.7 |
| 2B | 3 | AVG | 5.7 | 5.0 | 2.5 | 2.8 | 3.3 | 3.5 | 3.5 |
| | | SD | 1.4 | 0.9 | 1.0 | 1.2 | 1.0 | 0.8 | 1.2 |
| | | RSD | 24.1 | 17.9 | 42.0 | 41.3 | 31.0 | 23.9 | 35.0 |
| 2F | 3 | AVG | 5.5 | 5.2 | 2.2 | 2.8 | 3.0 | 3.5 | 2.6 |
| | | SD | 1.0 | 1.0 | 1.2 | 1.2 | 1.4 | 0.8 | 0.5 |
| | | RSD | 19.1 | 19.0 | 54.0 | 41.3 | 47.1 | 23.9 | 21.1 |

| | | | | Compared | Compared | Compared | | | |
|---|---|---|---|---|---|---|---|---|---|
| | | | | to | to | to | | | |
| Example | Ideal | Overall | | Usual E-liq | Usual E-Liq | Usual E-Liq | As | Purchase | |
| # | Aftertaste | Taste | Aftertaste | (Satisfaction) | (Differs) | (Better [symbol] | Replacement | Intent | Overall |
| 3 | 3.1 | 4.0 | 4.1 | 2.1 | 3.1 | 3.0 | 1.6 | 3.5 | 67.5 |
| (Control) | 1.0 | 0.8 | 0.8 | 0.6 | 1.1 | 0.8 | 0.5 | 1.3 | 18.3 |
| 2N | 31.7 | 18.9 | 20.2 | 30.2 | 36.0 | 25.2 | 31.8 | 37.4 | 27.1 |
| | 3.1 | 4.3 | 3.6 | 1.3 | 3.5 | 2.5 | 1.5 | 3.0 | 65.0 |
| | 1.5 | 0.5 | 0.7 | 0.5 | 0.9 | 0.9 | 0.5 | 0.8 | 12.0 |
| | 46.6 | 10.9 | 20.5 | 37.0 | 26.5 | 37.0 | 35.6 | 25.2 | 18.4 |
| 2P | 3.0 | 3.9 | 3.1 | 1.2 | 3.4 | 2.8 | 1.4 | 2.8 | 61.7 |
| | 0.9 | 0.6 | 0.6 | 0.4 | 0.7 | 0.8 | 0.5 | 1.0 | 15.0 |
| | 28.9 | 16.5 | 20.5 | 36.1 | 21.1 | 30.0 | 37.6 | 35.0 | 24.3 |
| 2Q | 2.4 | 4.4 | 4.1 | 1.5 | 3.3 | 3.3 | 2.1 | 4.1 | 76.9 |
| | 0.7 | 0.7 | 0.8 | 0.5 | 1.0 | 1.0 | 1.2 | 0.8 | 9.6 |
| | 31.3 | 17.0 | 20.2 | 35.6 | 31.8 | 31.8 | 58.7 | 20.2 | 12.5 |
| 2R | 2.9 | 4.3 | 4.0 | 1.6 | 3.1 | 2.8 | 1.6 | 3.3 | 68.1 |
| | 1.2 | 0.7 | 0.8 | 0.5 | 1.0 | 0.7 | 0.5 | 0.9 | 15.3 |
| | 43.4 | 16.6 | 18.9 | 31.8 | 31.7 | 25.7 | 31.8 | 27.3 | 22.5 |
| 2S | 2.5 | 4.3 | 3.9 | 2.0 | 2.9 | 3.6 | 2.0 | 4.1 | 76.9 |
| | 0.5 | 0.7 | 0.8 | 0.5 | 1.0 | 1.1 | 0.5 | 1.1 | 15.1 |
| | 21.4 | 16.6 | 21.5 | 26.7 | 34.5 | 29.3 | 26.7 | 27.3 | 19.6 |
| 2T | 2.7 | 4.2 | 3.6 | 2.4 | 3.2 | 3.4 | 2.0 | 4.2 | 81.0 |
| | 1.4 | 0.8 | 0.5 | 0.5 | 0.4 | 0.9 | 0.0 | 0.8 | 9.6 |
| | 51.2 | 19.9 | 15.2 | 22.8 | 14.0 | 26.3 | 0.0 | 19.9 | 11.9 |
| 2W | 3.4 | 4.8 | 4.4 | 1.6 | 4.0 | 4.0 | 2.0 | 3.8 | 75.0 |
| | 1.1 | 0.4 | 0.9 | 0.5 | 0.7 | 0.7 | 0.0 | 0.8 | 8.7 |
| | 33.5 | 9.3 | 20.3 | 34.2 | 17.7 | 17.7 | 0.0 | 22.0 | 11.5 |
| 2I | 3.6 | 4.6 | 4.2 | 2.2 | 3.4 | 3.8 | 2.0 | 4.6 | 76.0 |
| | 0.9 | 0.5 | 0.4 | 0.8 | 1.1 | 0.8 | 0.7 | 0.5 | 8.9 |
| | 24.8 | 11.9 | 10.6 | 38.0 | 33.5 | 22.0 | 35.4 | 11.9 | 11.8 |
| 2S | 2.5 | 3.5 | 3.3 | 1.6 | 3.3 | 2.8 | 1.5 | 3.2 | 62.5 |
| | 0.8 | 0.8 | 0.8 | 0.5 | 1.0 | 0.8 | 0.5 | 1.0 | 14.7 |
| | 33.5 | 23.9 | 24.5 | 34.2 | 31.0 | 29.9 | 36.5 | 31.0 | 23.6 |
| 2S | 2.3 | 4.0 | 3.7 | 1.7 | 3.3 | 2.7 | 2.0 | 3.4 | 70.0 |
| | 1.0 | 0.6 | 0.8 | 0.5 | 0.8 | 0.8 | 0.0 | 0.5 | 13.7 |
| | 44.3 | 15.8 | 22.3 | 31.0 | 24.5 | 30.6 | 0.0 | 16.1 | 19.6 |
| 2D | 1.8 | 4.0 | 4.2 | 2.0 | 4.2 | 2.8 | 1.8 | 3.6 | 70.0 |
| | 0.4 | 1.0 | 0.8 | 0.7 | 0.8 | 0.8 | 0.8 | 1.3 | 20.0 |
| | 24.8 | 25.0 | 19.9 | 35.4 | 19.9 | 29.9 | 46.5 | 37.3 | 28.6 |
| 2D | 2.0 | 4.2 | 4.0 | 2.0 | 3.8 | 3.4 | 2.0 | 4.3 | 68.0 |
| | 0.7 | 0.4 | 0.7 | 0.0 | 0.4 | 0.5 | 0.0 | 1.0 | 22.0 |
| | 35.4 | 10.6 | 17.7 | 0.0 | 11.8 | 16.1 | 0.0 | 22.5 | 32.3 |
| 2E | 3.2 | 3.5 | 3.8 | 1.8 | 4.0 | 3.5 | 1.7 | 3.3 | 70.0 |
| | 1.0 | 0.8 | 0.4 | 0.4 | 0.6 | 0.8 | 0.5 | 1.0 | 12.6 |
| | 31.0 | 23.9 | 10.6 | 22.3 | 15.8 | 23.9 | 31.0 | 31.0 | 18.1 |
| 2E | 3.4 | 3.4 | 3.2 | 1.6 | 3.4 | 2.4 | 1.6 | 2.6 | 59.0 |
| | 0.9 | 0.5 | 0.4 | 0.5 | 1.1 | 0.5 | 0.5 | 0.5 | 5.5 |
| | 26.3 | 16.1 | 14.0 | 34.2 | 33.5 | 22.8 | 34.2 | 21.1 | 9.3 |
| 2L | 2.7 | 4.3 | 3.8 | 2.5 | 4.0 | 3.2 | 2.2 | 3.5 | 71.7 |
| | 1.4 | 0.8 | 1.2 | 0.5 | 0.9 | 1.1 | 0.8 | 1.0 | 12.9 |
| | 51.2 | 18.8 | 30.5 | 21.9 | 22.4 | 34.2 | 34.7 | 30.0 | 18.0 |
| 2L | 2.7 | 4.3 | 3.7 | 1.7 | 3.5 | 2.8 | 2.0 | 3.5 | 69.2 |
| | 1.4 | 0.5 | 1.0 | 0.5 | 1.0 | 0.8 | 0.6 | 0.5 | 9.2 |
| | 51.2 | 11.9 | 28.2 | 31.0 | 30.0 | 26.6 | 31.6 | 15.6 | 13.3 |
| 2V | 2.5 | 3.2 | 3.2 | 1.7 | 4.0 | 2.5 | 1.3 | 3.2 | 64.2 |
| | 0.5 | 1.0 | 0.8 | 0.5 | 0.6 | 0.8 | 0.5 | 1.0 | 19.1 |
| | 21.9 | 31.0 | 23.8 | 31.0 | 15.8 | 33.5 | 38.7 | 31.0 | 29.7 |
| 2C | 3.3 | 4.0 | 3.5 | 2.2 | 3.7 | 3.3 | 1.8 | 3.5 | 70.8 |
| | 1.4 | 1.1 | 1.2 | 0.4 | 0.8 | 1.5 | 0.8 | 1.5 | 18.6 |
| | 41.0 | 27.4 | 35.0 | 20.3 | 22.3 | 45.2 | 41.1 | 43.3 | 26.2 |
| 2M | 3.4 | 3.8 | 4.0 | 1.8 | 4.0 | 3.0 | 1.8 | 3.2 | 59.0 |
| | 1.5 | 0.8 | 0.7 | 0.8 | 0.7 | 1.4 | 0.8 | 1.6 | 21.6 |
| | 44.6 | 22.0 | 17.7 | 46.5 | 17.7 | 47.1 | 46.5 | 51.3 | 36.6 |
| 2G | 2.2 | 4.2 | 3.8 | 2.0 | 4.0 | 3.4 | 2.0 | 4.4 | 74.0 |
| | 0.4 | 0.4 | 0.4 | 0.7 | 0.0 | 0.9 | 0.0 | 0.5 | 15.6 |
| | 20.3 | 10.6 | 11.8 | 35.4 | 0.0 | 26.3 | 0.0 | 12.4 | 21.0 |
| 2K | 2.8 | 3.4 | 3.6 | 2.0 | 4.0 | 3.2 | 2.0 | 3.8 | 68.0 |
| | 0.4 | 1.3 | 1.1 | 0.7 | 0.0 | 1.3 | 0.7 | 1.3 | 17.9 |
| | 16.0 | 39.5 | 31.7 | 35.4 | 0.0 | 40.7 | 35.4 | 34.3 | 26.3 |
| 2J | 2.8 | 4.2 | 3.8 | 2.2 | 3.6 | 3.4 | 2.0 | 3.8 | 72.0 |
| | 0.4 | 1.3 | 1.1 | 0.4 | 0.5 | 1.3 | 0.7 | 1.3 | 22.5 |
| | 16.0 | 31.0 | 28.8 | 20.3 | 15.2 | 39.5 | 35.4 | 34.3 | 31.3 |
| 2U | 2.9 | 3.6 | 3.4 | 1.7 | 4.3 | 2.9 | 1.4 | 3.0 | 56.4 |
| | 1.3 | 1.3 | 1.3 | 0.5 | 0.5 | 1.2 | 0.5 | 1.3 | 19.7 |
| | 47.1 | 35.6 | 37.1 | 28.5 | 11.4 | 42.5 | 37.4 | 43.0 | 35.0 |
| | 2.7 | 3.1 | 3.0 | 1.7 | 4.0 | 2.9 | 1.4 | 3.1 | 55.7 |
| | 1.3 | 1.5 | 1.0 | 0.5 | 0.6 | 1.2 | 0.5 | 1.6 | 27.8 |
| | 46.2 | 46.6 | 33.3 | 28.5 | 14.4 | 42.5 | 37.4 | 50.1 | 49.8 |
| 2A | 2.5 | 3.5 | 3.7 | 2.2 | 3.8 | 3.3 | 1.8 | 3.2 | 62.5 |
| | 1.4 | 1.4 | 1.4 | 0.8 | 0.8 | 1.2 | 0.8 | 1.3 | 18.9 |
| | 55.1 | 39.4 | 37.3 | 34.7 | 19.6 | 36.3 | 41.1 | 42.0 | 30.3 |
| 2H | 2.7 | 4.5 | 4.0 | 2.2 | 4.2 | 4.2 | 2.0 | 4.7 | 82.5 |
| | 1.2 | 0.5 | 1.0 | 0.4 | 0.8 | 0.4 | 0.0 | 0.8 | 6.1 |
| | 45.4 | 12.2 | 25.0 | 18.8 | 18.1 | 10.6 | 0.0 | 17.5 | 7.4 |
| 2B | 2.5 | 3.7 | 3.8 | 2.0 | 3.7 | 3.3 | 1.8 | 3.3 | 65.0 |
| | 1.4 | 1.2 | 1.0 | 0.9 | 0.5 | 1.2 | 1.0 | 1.6 | 22.8 |
| | 55.1 | 33.0 | 25.6 | 44.7 | 14.1 | 36.3 | 53.6 | 49.0 | 35.1 |
| 2F | 2.5 | 3.5 | 3.5 | 1.7 | 3.5 | 2.7 | 1.6 | 3.3 | 60.8 |
| | 1.4 | 0.8 | 0.8 | 0.5 | 0.5 | 1.2 | 0.5 | 1.0 | 21.5 |
| | 55.1 | 23.9 | 23.9 | 31.0 | 15.6 | 45.4 | 31.0 | 31.0 | 35.4 |

[symbol]

indicates data missing or illegible when filed

1. A liquid aerosolizable composition, comprising:
an aerosol former; and
an active agent selected from:
(i) a substituted 3-(1-methylpyrrolidin-2-yl)pyridine having a structure according to Formula I: 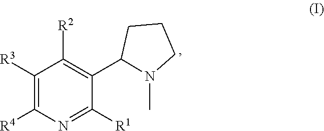
wherein:
R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted, and at least one of R¹, R², R³, and R⁴ are not hydrogen;
(ii) a substituted 3-(1-methylpyrrolidin-2-yl)pyridine having a structure according to Formula II: 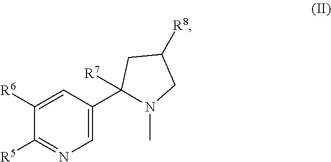
wherein R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen; and
(iii) a 3-(azetidin-2-yl)pyridine or 3-(azetidin-2-ylmethoxy)pyridine having a structure according to Formula III: 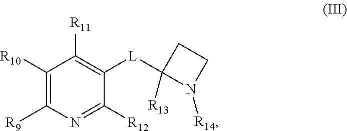
wherein L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

2. The composition of claim 1, wherein the active agent has a structure according to Formula I, and wherein R¹, R², and R³ are each H.

3. The composition of claim 1, wherein the active agent has a structure according to Formula I, and wherein R⁴ is optionally substituted C₁-C₆ alkyl, F, Cl, Br, OCH₃, OEt, or CN.

4. The composition of claim 1, wherein the active agent has a structure according to Formula I, and wherein R¹, R², and R³ are each H, and R⁴ is C₁-C₃ alkyl, such as wherein R⁴ is CH₃.

5. The composition of claim 1, wherein the active agent has a structure according to Formula I, and wherein the substituted 3-(1-methylpyrrolidin-2-yl)pyridine according to Formula I is racemic.

6. The composition of claim 1, wherein the active agent has a structure according to Formula I, and wherein the substituted 3-(1-methylpyrrolidin-2-yl)pyridine according to Formula I has an (R)-configuration or an (S)-configuration, such as wherein the substituted 3-(1-methylpyrrolidin-2-yl)pyridine has an (S)-configuration.

7. The composition of claim 1, wherein the composition is substantially free of 3-(1-methylpyrrolidin-2-yl)pyridine, such as wherein the composition is completely free of 3-(1-methylpyrrolidin-2-yl)pyridine.

8. The composition of claim 1, wherein the active agent has a structure according to Formula I, and wherein the substituted 3-(1-methylpyrrolidin-2-yl)pyridine according to Formula I is present in an amount from about 0.1 to about 5% by weight of the composition, calculated as the free base and based on the total weight of the composition, such as about 0.1 to about 1.0% by weight or about 0.1 to 0.75% by weight or about 0.1 to about 0.5% by weight.

9. The composition of claim 1, wherein the composition is substantially free of acid components; optionally, wherein the composition is completely free of acid components.

10. The composition of claim 1, further comprising an organic acid.

11. The composition of claim 10, wherein the active agent has a structure according to Formula I, and wherein at least a portion of the substituted 3-(1-methylpyrrolidin-2-yl)pyridine according to Formula I is present in the form of a salt with the organic acid.

12. The composition of claim 10, wherein the active agent has a structure according to Formula I, and wherein at least a portion of the substituted 3-(1-methylpyrrolidin-2-yl)pyridine according to Formula I is present in the form of a salt with tartaric acid.

13. The composition of claim 10, wherein the active agent has a structure according to Formula I, and wherein at least a portion of the substituted 3-(1-methylpyrrolidin-2-yl)pyridine according to Formula I is present in the form of a salt with benzoic acid, lactic acid, levulinic acid, or a combination thereof.

14. The composition of claim 10, wherein the organic acid and the substituted 3-(1-methylpyrrolidin-2-yl)pyridine of Formula I are present in a molar ratio in a range from about 1:10 to about 2:1, such as about 1:2 to about 2:1, or about 0.9:1 to about 1.1:1, such as wherein the molar ratio is 1:10 to about 1:1 or about 1:10 to about 1:1.1 or about 1:5 to about 1:1.2 or about 1:4 to about 1:1.5.

15. The composition of claim 1, wherein the aerosol former comprises one or more polyhydric alcohols, water, or a combination thereof; optionally, wherein the one or more polyhydric alcohols is selected from the group consisting of glycerol, propylene glycol, 1,3-propanediol, diethylene glycol, triethylene glycol, a polyethylene glycol, and combinations thereof, such as wherein the polyhydric alcohol is selected from the group consisting of glycerol, propylene glycol, and mixtures thereof, or wherein the aerosol former comprises propylene glycol and glycerol.

16. The composition of claim 15, wherein the aerosol former comprises propylene glycol and glycerol in a ratio by weight from about 1:3 to about 3:1; such as about 1:1 to about 2.5:1 or about 1.1:1 to about 2:1 or about 1.1:1 to about 1.5:1.

17. An aerosol delivery device comprising a housing enclosing a chamber containing the liquid aerosolizable composition of claim 1.

18. An aerosol delivery device comprising:
a housing enclosing a chamber containing the liquid aerosolizable composition of claim 1;
an atomizer in fluid communication with the chamber and configured to aerosolize the liquid aerosolizable composition to form an aerosol; and
an aerosol pathway positioned to carry the aerosol to a mouth-end of the aerosol delivery device.

19. The aerosol delivery device of claim 18, further comprising a power source electronically connected to the atomizer.

20. The aerosol delivery device of claim 19, further comprising a controller configured to control the power transmitted by the power source to the atomizer.

21. The aerosol delivery device of claim 18, wherein the atomizer comprises a heating element or a piezoelectric element.

22. A process for forming an aerosol, the process comprising aerosolizing a liquid aerosolizable composition of claim 1.

23. An aerosol comprising:
an aerosol former; and
an active agent selected from:
(i) a substituted 3-(1-methylpyrrolidin-2-yl)pyridine having a structure according to Formula I: 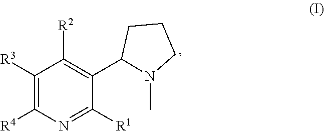
wherein:
R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted, and at least one of R¹, R², R³, and R⁴ are not hydrogen;
(ii) a substituted 3-(1-methylpyrrolidin-2-yl)pyridine having a structure according to Formula II: 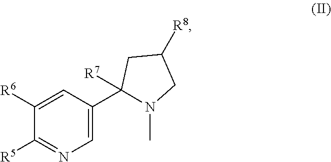
wherein R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R¹ and R⁸ is not hydrogen; and
(iii) a 3-(azetidin-2-yl)pyridine or 3-(azetidin-2-ylmethoxy)pyridine having a structure according to Formula III: 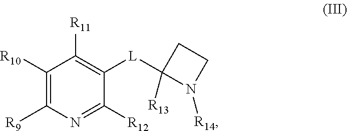
wherein L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.
